(19) United States
(12) Patent Application Publication
(10) Pub. No.: US 2023/0094830 A1
(43) Pub. Date: Mar. 30, 2023

(54) SIGNATURES FOR PREDICTING CANCER IMMUNE THERAPY RESPONSE

(71) Applicant: Myriad Genetics, Inc., Salt Lake City, UT (US)

(72) Inventor: Susanne WAGNER, Salt Lake City, UT (US)

(73) Assignee: Myriad Genetics, Inc., Salt Lake City, UT (US)

(21) Appl. No.: 18/072,520

(22) Filed: Nov. 30, 2022

Related U.S. Application Data
(63) Continuation of application No. 15/982,126, filed on May 17, 2018.
Application No. 15/982,126 is a continuation of application No. PCT/US2016/062807, filed on Nov. 18, 2016.
(60) Provisional application No. 62/257,299, filed on Nov. 19, 2015.
(60) Provisional application No. 62/259,520, filed on Nov. 24, 2015.
(60) Provisional application No. 62/259,477, filed on Nov. 24, 2015.

(51) Int. Cl. G01N 33/574; G16B 30/00; C12Q 1/6886; G16H 50/30; G16H 50/20; G01N 33/573

(52) U.S. Cl. CPC G01N 33/574; G16B 30/00; C12Q 1/6886; G16H 50/30; G16H 50/20; G01N 33/573; C12Q 2600/158; C12Q 2600/156; C12Q 2600/106; C12Q 1/686

(57) ABSTRACT

This disclosure generally relates to a molecular classification of cancer and particularly to molecular markers for predicting response to cancer therapy, including cancer immune therapy, and methods of use thereof.

### RELATED APPLICATIONS

This application is a continuation of and claims priority benefit of International Application serial number PCT/US2016/062807 filed Nov. 18, 2016, which in turn claims the priority benefit of U.S. provisional applications Ser. No. 62/257,299 (filed Nov. 19, 2015), 62/259,520 (filed Nov. 24, 2015), and 62/259,477 (filed Nov. 24, 2015), the entire contents of each of which are hereby incorporated by reference.

### FIELD OF THE DISCLOSURE

This disclosure generally relates to a molecular classification of cancer and particularly to molecular markers for predicting response to cancer therapy, including cancer immune therapy, and methods of use thereof.

### BACKGROUND OF THE DISCLOSURE

Cancer is a major public health problem, accounting for roughly 25% of all deaths in the United States. American Cancer Society, FACTS AND FIGURES 2010. Though many treatments have been devised for various cancers, these treatments often vary in severity of side effects. One class of cancer therapeutics that has shown recent promise is often referred to as immune checkpoint inhibitors. Snyder et al., N. ENGL. J. MED. (2014) 371:2189-2199. However, there is a significant unmet need for molecular diagnostic tools for detecting and/or predicting response or resistance to such therapeutics.

### SUMMARY OF THE DISCLOSURE

This document describes the development of gene panels for classifying cancer. Classifying cancer using these signatures generally includes prediction of response to, or selection of, particular therapeutic treatments or regimens. In particular, the studies described herein allowed for the development of sets or panels of genes related to antigen processing (herein referred to as “antigen processing machinery genes” or “APM genes”), which panels can be utilized and applied in laboratory methods for predicting response to particular classes of cancer therapies. These APM genes include, but are not limited to, HLA class II activation-related genes (“HLAGs” or “HLAG” in the singular), which were found to be mutated in cancer cells from patients and shown in these studies to be useful in laboratory methods for predicting therapy response.

Accordingly, in one aspect, the present disclosure provides a method for detecting mutations in a panel of genes in a sample from a patient identified as having cancer. Generally, the method includes at least the following steps: (1) obtaining, or providing, one or more samples from a patient identified as having cancer; and (2) assaying the sample to determine or detect the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3; wherein (a) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (b) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

In another aspect, the present disclosure provides a method for treating cancer patients comprising: (1) assaying one or more patient samples comprising or derived from a cancer cell to determine or detect the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3; (2) determining whether any of the test genes harbors a mutation; and (3)(a) recommending, prescribing or administering a treatment regimen comprising an immune checkpoint inhibitor to a patient in whose sample no test gene is determined in (2) to harbor a mutation or (3)(b) recommending, prescribing or administering a treatment regimen not comprising an immune checkpoint inhibitor to a patient in whose sample at least one test gene is determined in (2) to harbor a mutation. In some embodiments (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes

In another aspect, the present disclosure provides a method for detecting resistance (and/or an increased likelihood of resistance) to a treatment regimen comprising an immune checkpoint inhibitor, the method comprising: (1) assaying one or more patient samples comprising or derived from a cancer cell to determine or detect the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3; (2) determining whether any of the test genes harbors a mutation; and (3)(a) recording in a tangible medium that a patient in whose sample at least one test gene is determined in (2) to harbor a mutation has an increased likelihood of resistance to a treatment regimen comprising an immune checkpoint inhibitor or (3)(b) recording in a tangible medium that a patient in whose sample no test gene is determined in (2) to harbor a mutation has a decreased likelihood of resistance to a treatment regimen comprising an immune checkpoint inhibitor. In some embodiments (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

The present disclosure further provides a system for detecting resistance (and/or an increased likelihood of resistance) to a treatment regimen comprising an immune checkpoint inhibitor, the system comprising: (1) a sample analyzer for assaying one or more patient samples comprising or derived from a cancer cell to determine or detect the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3, wherein the sample analyzer contains the sample or DNA molecules extracted or derived from the sample; (2) a first computer program for receiving test genetic sequence data on the test genes; (3) a second computer program for comparing the test genetic sequence data to one or more reference genetic sequences for each test gene to determine whether any of the test genes harbors a mutation; and (4) a third computer program for determining (a) that a patient in whose sample at least one test gene is determined by the second computer program in (3) to harbor a mutation has an increased likelihood of resistance to a treatment regimen comprising an immune checkpoint inhibitor or (b) that a patient in whose sample no test gene is determined by the second computer program in (2) to harbor a mutation has a decreased likelihood of resistance to a treatment regimen comprising an immune checkpoint inhibitor. In some embodiments, the system further comprises a display module displaying the comparison between the test sequence(s) and the reference sequence(s), or displaying a result of the computerized comparison.

The present disclosure further provides a diagnostic kit for detecting resistance (and/or an increased likelihood of resistance) to a treatment regimen comprising an immune checkpoint inhibitor, the kit comprising, in a compartmentalized container, a plurality of oligonucleotides hybridizing to a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes. The kit may further include one or more oligonucleotides hybridizing to one or more control genes. The oligonucleotides can be hybridizing probes for hybridization with an amplification product of the gene(s) (e.g., an amplification product of DNA corresponding to the gene) under stringent conditions or primers suitable for PCR amplification of the genes (e.g., suitable for amplification of DNA of a sample obtained from, e.g., fresh tumor tissue or FFPE tumor tissue). Either the probes or the primers may be labelled (e.g., with a fluorescent tag). In one embodiment, the kit consists essentially of, in a compartmentalized container, a plurality of PCR reaction mixtures for PCR amplification of DNA from a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes, and wherein each reaction mixture comprises a PCR primer pair for PCR amplifying DNA that corresponds to one of the test genes. In some embodiments the kit includes instructions for detecting resistance (and/or an increased likelihood of resistance) to a treatment regimen comprising an immune checkpoint inhibitor based at least in part on the presence or absence of mutations in the test genes. In some embodiments the kit comprises one or more computer software programs for detecting resistance (and/or an increased likelihood of resistance) to a treatment regimen comprising an immune checkpoint inhibitor based at least in part on the presence or absence of mutations in the test genes. In some embodiments such computer software is programmed to communicate (e.g., display) or to instruct a computer to record in a tangible medium whether (a) a patient in whose sample at least one test gene is determined in (2) to have a mutation has an increased likelihood of resistance to a treatment regimen comprising an immune checkpoint inhibitor or (b) a patient in whose sample no test gene is determined in (2) to have a mutation has a decreased likelihood of resistance to a treatment regimen comprising an immune checkpoint inhibitor. In one aspect, the kit includes reagents necessary for extracting DNA from fresh tumor tissue, fresh frozen tumor tissue, or FFPE tumor tissue.

The present disclosure also provides the use of (1) a plurality of oligonucleotides hybridizing to a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes; for determining the sequence of the test genes in a sample from a patient having cancer, for detecting resistance (and/or an increased likelihood of resistance) to a treatment regimen comprising an immune checkpoint inhibitor, wherein (a) the presence of a mutation in at least one test gene indicates an increased likelihood of resistance and (b) no detected mutation in any test gene indicates no increased likelihood of resistance. In some embodiments, the oligonucleotides are PCR primers suitable for PCR amplification of the test genes. Either the probes or the primers may be labelled (e.g., with a fluorescent tag). In other embodiments, the oligonucleotides are probes hybridizing to DNA that corresponds to the test genes under stringent conditions. In some embodiments, the plurality of oligonucleotides are probes for hybridization under stringent conditions to, or are suitable for PCR amplification of DNA that corresponds to a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

Unless otherwise defined, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. In case of conflict, the present specification, including definitions, will control. In addition, the materials, methods, and examples are illustrative only and not intended to be limiting.

Other features and advantages of the disclosure will be apparent from the following Detailed Description, and from the Claims.

### DESCRIPTION OF THE DRAWINGS

FIG. 1 summarizes the types and frequency of mutations in APM genes in particular cancer types.

FIG. 2 summarizes the types and frequency of mutations in specific APM genes in melanoma.

FIG. 3 summarizes the types and frequency of mutations in specific APM genes in squamous lung cancer.

FIG. 4 summarizes the types and frequency of mutations in specific APM genes in lung adenocarcinoma.

FIG. 5 summarizes the types and frequency of mutations in specific APM genes in gastric cancer.

FIG. 6 summarizes the types and frequency of mutations in specific APM genes in head and neck squamous cell cancer.

FIG. 7 summarizes the types and frequency of mutations in specific APM genes in prostate cancer.

### DETAILED DESCRIPTION OF THE DISCLOSURE

### A. Definitions

As used in this disclosure, “antigen processing machinery gene” or “APM gene” refers to one of a group of genes with a role in the antigen processing machinery of the cell. APM genes include the genes listed in Table 1 or Table 3, including HLAGs (as defined below) and non-HLA related genes.

As used in this disclosure, “immune checkpoint inhibitor” refers to a therapeutic agent whose mode of action is to prevent (or inhibit) immune cells and/or the immune response from being turned off (or down-regulated or inhibited) by cancer cells. Examples include the therapeutic agents listed in Table 2 below.

As used in this disclosure, “sample” or “biological sample” refers to an amount of tissue or bodily fluid taken from a subject, such as a human patient, or any biomolecule derived therefrom. Biomolecules derived from a tissue or fluid include molecules originally present in such tissue or fluid and extracted therefrom as well as artificial counterparts synthesized based on such endogenous biomolecules. Non-limiting examples of artificial counterparts include PCR products using endogenous nucleic acids as templates (e.g., cDNA synthesized from mRNA, PCR amplification of genomic DNA or cDNA, etc.). Non-limiting examples of bodily fluids include urine, blood, plasma, serum, semen, perspiration, tears, mucus, and tissue lystates. A “sample” or “biological sample” further refers to a homogenate, lysate, or extract prepared from a subject's tissues, cells, or component parts, or a fraction or portion thereof. A “sample” or “biological sample” can refer to non-cellular biological material, such as blood or urine.

In some embodiments of the various aspects of the present disclosure the “sample” is a “tumor sample.” As used herein, “tumor sample” refers to any sample containing one or more tumor cells, or tumor-derived DNA, RNA or protein, and obtained from an individual currently or previously diagnosed with cancer, an individual undergoing cancer treatment, or an individual not diagnosed with cancer but who presents with symptoms consistent with a cancer diagnosis. For example, a tissue sample obtained from a tumor tissue of an individual is a useful tumor sample in the present disclosure. The tissue sample can be a formalin-fixed, paraffin-embedded (FFPE) sample, or fresh frozen sample, and preferably contain largely tumor cells. A single malignant cell from a patient's tumor is also a useful tumor sample. Such a malignant cell can be obtained directly from the patient's tumor, or purified from the patient's bodily fluid (e.g., blood, urine). Thus, a bodily fluid such as blood, urine, sputum and saliva containing one or tumor cells, or tumor-derived DNA, RNA or proteins, can also be useful as a tumor sample for purposes of practicing the present disclosure

As used in this disclosure, “mutation” refers to a variation in a patient's gene sequence from the expected (or a reference) gene sequence, wherein such variation is known or predicted to reduce or abolish the normal activity of the gene. One example is missense mutations, which alter the sequence of amino acids in the protein encoded by a test gene of the disclosure by converting the original codon encoding a first amino acid to a mutant codon encoding a second amino acid that is different from the first amino acid, and where this amino acid change is expected to reduce or abolish the normal activity of the encoded protein. Another example is truncating mutations, which result in a truncation of the protein encoded by the gene and can include nonsense mutations (where a single base change converts an amino acid encoding codon to a stop codon) and frameshift mutations (where insertion or deletion of one, two, or more (typically a multiple of one or two but not three) nucleotides alters the normal or native reading frame of the codons that make up the coding sequence of the mRNA transcript of the gene). These frameshift mutations can result in truncations of the encoded protein since altered reading frames can contain stop codons that will be encountered by the translational machinery of the cell in advance of the native stop codon. Frameshift mutations that result in altered reading frames can also result in a different sequence of amino acids being added to the carboxyl-terminus of a protein as a result of the translational machinery translating in a different reading frame before encountering a stop codon in this new frame. Another example is splicing mutations, which adversely alter the splicing of exons, or the removal of introns, from the transcript transcribed from a diagnostic gene of the disclosure (typically by occurring at or near one of the so-called “splice junctions” that are found at the boundaries of the encoded exons and the introns that separate them). Such mutations can cause alterations in the amino acid sequence and structure of the protein encoded by a test gene of the disclosure. Alternatively, such mutations result in truncations of the encoded protein, because stop codons can occur in multiple reading frames.

As used in this disclosure, “administering a treatment regimen” refers to providing a patient with the treatment regimen, non-limiting examples of which include injecting (e.g., subcutaneous, intravenous, intraperitoneal, etc.) a therapeutic agent into a patient's body, applying a therapeutic agent topically to a patient's skin, inserting a therapeutic agent into a patient's mouth or anus, etc. As used in this disclosure, “recommending a treatment regimen” refers to providing a suggestion, including but not limited to one suggestion amongst a plurality of suggestions, that a patient may consider self-administering, or having administered to the patient, the treatment regimen. Such a suggestion may include listing the treatment regimen in a list of suggested options, highlighting the treatment regimen amongst the options, promoting the treatment regimen as preferred, etc. As used in this disclosure, “prescribing a treatment regimen” refers to providing a patient with an order or other instructions that the treatment regimen be administered.

As used in this disclosure, “resistance to a treatment regimen” refers to absence of response to initial administration of the regimen or subsequent loss or significant diminution of response to the regimen. Response can be measured clinically (e.g., by gross physical examination), by pathology (e.g., imaging or other test to measure tumor size, tumor cell characteristics, etc.), biochemically (e.g., by assessment of one or more biomarkers indicative of response), etc.

As used in this disclosure, “recording [e.g., information] in a tangible medium” refers to capturing information in a physical structure (1) capable of storing such information and (2) enabling retrieval of such information. Examples of physical structures include paper, computer hardware (e.g., hard disk drives, flash memory), etc.

As used in this disclosure, “probe” and “oligonucleotide” (also “oligo”), when used in the context of nucleic acids, interchangeably refer to a relatively short nucleic acid fragment or sequence. The disclosure also provides primers useful in the methods of the disclosure. “Primers” are probes capable, under the right conditions and with the right companion reagents, of selectively amplifying a target nucleic acid (e.g., a target gene). In the context of nucleic acids, “probe” is used herein to encompass “primer” since primers can generally also serve as probes.

Unless expressly stated otherwise, every mention in this disclosure of any given “method” hereby expressly includes an “in vitro method”. Such “in vitro methods” generally refer to methods not practiced directly on the human body, though they may involve the use of materials (e.g., samples) obtained from the human body.

### B. Immune System Genes Useful in the Disclosure

This document describes development of gene panels useful in methods, kits and systems for predicting response to a particular class of therapeutic agents. In particular, genes related to antigen processing (herein referred to as “antigen processing machinery genes” or “APM genes”) were identified as predictive of response to immune checkpoint inhibitor agents. Specific sets of genes shown to be useful, individually and as one or more panels, are listed in Table 1 and Table 3.

The genes identified in these studies include immune system genes, or APM genes, that for convenience can further be subdivided into two subgroups based on their general biological characteristics: HLA related genes (“HLAGs”; gene numbers 1-6 in Table 1 below) and non-HLA related genes (gene numbers 7-15 in Table 1 below). These genes are shown herein to be very useful in laboratory methods for detecting and/or predicting resistance (or an increased likelihood of resistance) to immune checkpoint inhibitor therapeutic agents.

TABLE 1
APM genes

| | | NCBI | | Chromosomal Location | | |
|---|---|---|---|---|---|---|
| Gene | Gene | Gene ID | | (NCBI Homo sapiens | | |
| # | Name | # | Description | Annotation Release 108) | Aliases | MIM |
| 1 | HLA-A | ID: 3105 | major | Chromosome 6, | HLAA | 142800 |
| | | | histocompatibility | NC_000006.12 | | |
| | | | complex, class I, A | (29942470 . . . 29945884) | | |
| 2 | HLA-B | ID: 3106 | major | Chromosome 6, | AS, B-4901, HLAB | 142830 |
| | | | histocompatibility | NC_000006.12 | | |
| | | | complex, class I, B | (31353866 . . . 31357245, | | |
| | | | | complement) | | |
| 3 | HLA-C | ID: 3107 | major | Chromosome 6, | D6S204, HLA-JY3, | 142840 |
| | | | histocompatibility | NC_000006.12 | HLAC, HLC-C, | |
| | | | complex, class I, C | (31268749 . . . 31272136, | MHC, PSORS1 | |
| | | | | complement) | | |
| 4 | HLA-E | ID: 3133 | major | Chromosome 6, | HLA-6.2, QA1 | 143010 |
| | | | histocompatibility | NC_000006.12 | | |
| | | | complex, class I, E | (30489406 . . . 30494205) | | |
| 5 | HLA-F | ID: 3134 | major | Chromosome 6, | CDA12, HLA-5.4, | 143110 |
| | | | histocompatibility | NC_000006.12 | HLA-CDA12, | |
| | | | complex, class I, F | (29723340 . . . 29740355) | HLAF | |
| 6 | HLA-G | ID: 3135 | major | Chromosome 6, | MHC-G | 142871 |
| | | | histocompatibility | NC_000006.12 | | |
| | | | complex, class I, G | (29826967 . . . 29831130) | | |
| 7 | B2M* | ID: 567 | beta-2- | Chromosome 15, | IMD43 | 109700 |
| | | | microglobulin | NC_000015.10 | | |
| | | | | (44711487 . . . 44718159) | | |
| 8 | CIITA* | ID: 4261 | class II major | Chromosome 16, | C2TAIV, | 600005 |
| | | | histocompatibility | NC_000016.10 | MHC2TA, | |
| | | | complex | (10866208 . . . 10941562) | NLRA, CIITA | |
| | | | transactivator | | | |
| 9 | ERAP1* | ID: 51752 | endoplasmic | Chromosome 5, | A-LAP, ALAP, | 606832 |
| | | | reticulum | NC_000005.10 | APPILS, ARTS-1, | |
| | | | aminopeptidase 1 | (96759245 . . . 96935983, | ARTSI, ERAAP, | |
| | | | | complement) | ERAAP1, PILS-AP, | |
| | | | | | PILSAP | |
| 10 | ERAP2* | ID: 64167 | endoplasmic | Chromosome 5, | L-RAP, LRAP | 609497 |
| | | | reticulum | NC_000005.10 | | |
| | | | aminopeptidase 2 | (96875940 . . . 96919703) | | |
| 11 | NLRC5* | ID: 84166 | NLR family CARD | Chromosome 16, | CLR16.1, NOD27, | 613537 |
| | | | domain containing | NC_000016.10 | NOD4 | |
| | | | 5 | (56989547 . . . 57083524) | | |
| 12 | PDIA3 | ID: 2923 | protein disulfide | Chromosome 15, | ER60, ERp57, | 602046 |
| | | | isomerase family | NC_000015.10 | ERp60, ERp61, | |
| | | | A member 3 | (43746392 . . . 43772606) | GRP57, GRP58, | |
| | | | | | HEL-S-269, HEL- | |
| | | | | | S-93n, HsT17083, | |
| | | | | | P58, PI-PLC | |
| 13 | TAP1* | ID: 6890 | transporter 1, ATP | Chromosome 6, | ABC17, ABCB2, | 170260 |
| | | | binding cassette | NC_000006.12 | APT1, D6S114E, | |
| | | | subfamily B | (32845209 . . . 32853971, | PSF-1, PSF1, | |
| | | | member | complement) | RING4*0102N, | |
| | | | | | TAP1N, TAP1 | |
| 14 | TAP2* | ID: 6891 | transporter 2, ATP | Chromosome 6, | ABC18, ABCB3, | 170261 |
| | | | binding cassette | NC_000006.12 | APT2, D6S217E, | |
| | | | subfamily B | (32821833 . . . 32838823, | PSF-2, PSF2, | |
| | | | member | complement) | RING11 | |
| 15 | TAPBP* | ID: 6892 | TAP binding | Chromosome 6, | NGS17, TAPA, | 601962 |
| | | | protein | NC_000006.12 | TPN, TPSN | |
| | | | | (33299694 . . . 33314387, | | |
| | | | | complement) | | |

Table 1 above provides a representative set of APM genes from which panels or signatures of the disclosure as described herein in the various embodiments and aspects of the disclosure (e.g., the sub-panel in Table 3) can be constructed.

### C. Methods of Detecting Mutations in APM Genes of the Disclosure

Accordingly, in one aspect, the present disclosure provides a method for detecting mutations in a panel of genes in a sample from a patient identified as having cancer. Generally, the method comprises the following steps: (1) obtaining, or providing, one or more samples obtained from a patient identified as having cancer; and (2) assaying the sample to determine the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3; wherein (a) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (b) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

### D. Methods of Measuring Expression in APM Genes of the Disclosure

Detecting an inactivating mutation in a gene is one way of detecting deficiency in that gene. Other biochemical causes may also result in abolished or reduced activity of a gene, including abolished or reduced expression of the RNA transcript encoded by such gene or reduced expression of the protein encoded by such RNA. Thus, as used throughout this disclosure, “deficiency” in a gene refers to a mutation in that gene or abolished or reduced expression of that gene's encoded RNA transcript or protein. Any disclosure herein of an embodiment of the invention involving a deficiency in a gene hereby expressly includes a description of at least two alternative sub-embodiments, one in which the deficiency is a mutation in the gene and another in which the deficiency is reduced expression of the gene.

Accordingly, in another aspect, the present disclosure provides a method for measuring expression of a panel of genes in a sample from a patient identified as having cancer. Generally, the method comprises the following steps: (1) obtaining, or providing, one or more samples obtained from a patient identified as having cancer; and (2) assaying the sample to determine the expression of each test gene in a panel of genes comprising at least 1, 2, 3, 4, 5, 6, 7, 8, 9, 10, 11, 12, 13, 14, or 15 test genes selected from the genes listed in Table 1 or Table 3; wherein (a) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (b) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

In some embodiments assaying the sample to determine the expression of a test gene comprises measuring the presence or amount of RNA transcripts of such gene (or cDNA reversed transcribed and/or amplified therefrom). In other embodiments, assaying the sample to determine the expression of a test gene comprises measuring the presence or amount of the protein(s) encoded by such gene.

### E. Methods of Using APM Genes of the Disclosure

In another aspect, the present disclosure provides a method for treating cancer patients comprising: (1) assaying one or more patient samples comprising or derived from a cancer cell to detect deficiency in a panel of genes comprising at least 1, 2, 3, 4, 5, 6, 7, 8, 9, 10, 11, 12, 13, 14, or 15 test genes selected from the genes listed in Table 1 or Table 3; (2) determining whether there is a deficiency in any of the test genes; and (3)(a) recommending, prescribing or administering a treatment regimen comprising an immune checkpoint inhibitor (e.g., a therapeutic agent listed in Table 2) to a patient in whose sample no deficiency is detected in any test gene in (2) or (3)(b) recommending, prescribing or administering a treatment regimen not comprising an immune checkpoint inhibitor (e.g., a therapeutic agent listed in Table 2) to a patient in whose sample at least one test gene is determined in (2) to have a deficiency.

In another aspect, the present disclosure provides a method for detecting resistance (and/or an increased likelihood of resistance) to a treatment regimen comprising an immune checkpoint inhibitor (e.g., a therapeutic agent listed in Table 2), the method comprising: (1) assaying one or more patient samples comprising or derived from a cancer cell to detect deficiency in a panel of genes comprising at least 1, 2, 3, 4, 5, 6, 7, 8, 9, 10, 11, 12, 13, 14, or 15 test genes selected from the genes listed in Table 1 or Table 3; (2) determining whether there is a deficiency in any of the test genes; and (3)(a) recording in a tangible medium that a patient in whose sample at least one test gene is determined in (2) to have a deficiency has an increased likelihood of resistance to a treatment regimen comprising an immune checkpoint inhibitor or (3)(b) recording in a tangible medium that a patient in whose sample no test gene is determined in (2) to have a deficiency has a decreased likelihood of resistance to a treatment regimen comprising an immune checkpoint inhibitor.

### F. Systems Using APM Genes of the Disclosure

Techniques for analyzing such expression, activity, and/or sequence data (indeed any data obtained according to the disclosure) may be implemented using hardware, software or a combination thereof in one or more computer systems or other processing systems capable of effectuating such analysis.

Thus, the present disclosure further provides a system for detecting resistance (and/or an increased likelihood of resistance) to a treatment regimen comprising an immune checkpoint inhibitor (e.g., a therapeutic agent listed in Table 2), the system comprising: (1) a sample analyzer for assaying one or more patient samples comprising or derived from a cancer cell to detect deficiency in a panel of genes comprising at least 1, 2, 3, 4, 5, 6, 7, 8, 9, 10, 11, 12, 13, 14, or 15 test genes selected from the genes listed in Table 1 or Table 3, wherein the sample analyzer contains the sample or DNA, RNA or protein molecules extracted or derived from the sample; (2) a first computer program for receiving test gene sequence, RNA expression, or protein expression data on the test genes; (3) a second computer program for comparing the data in (2) to one or more reference gene sequences, RNA expression data, or protein expression data for each test gene to determine whether any of the test genes harbors a deficiency; and (4) a third computer program for determining (a) that a patient in whose sample at least one test gene is determined by the second computer program in (3) to have a deficiency has an increased likelihood of resistance to a treatment regimen comprising an immune checkpoint inhibitor or (b) that a patient in whose sample no test gene is determined by the second computer program in (3) to have a deficiency has a decreased likelihood of resistance to a treatment regimen comprising an immune checkpoint inhibitor. In some embodiments, the system further comprises a display module displaying the comparison between the test sequence(s) and the reference sequence(s), or displaying a result of the computerized comparison.

The sample analyzer can be any instrument useful in detecting gene sequences or measuring gene expression, including, e.g., a sequencing machine (e.g., Illumina HiSeg™, Ion Torrent PGM, ABI SOLiD™ sequencer, PacBio RS, Helicos Heliscope™, etc.), a real-time PCR machine (e.g., ABI 7900, Fluidigm BioMark™, etc.), a microarray instrument, etc.

The computer-based analysis function can be implemented in any suitable language and/or browsers. For example, it may be implemented with C language and preferably using object-oriented high-level programming languages such as Visual Basic, SmallTalk, C++, and the like. The application can be written to suit environments such as the Microsoft Windows™ environment including Windows™ 98, Windows™ 2000, Windows™ NT, and the like. In addition, the application can also be written for the MacIntosh™, SUN™, UNIX or LINUX environment. In addition, the functional steps can also be implemented using a universal or platform-independent programming language. Examples of such multi-platform programming languages include, but are not limited to, hypertext markup language (HTML), JAVA™, JavaScript™, Flash programming language, common gateway interface/structured query language (CGI/SQL), practical extraction report language (PERL), AppleScript™ and other system script languages, programming language/structured query language (PL/SQL), and the like. Java™- or JavaScript™-enabled browsers such as HotJava™, Microsoft™ Explorer™, or Netscape™ can be used. When active content web pages are used, they may include Java™ applets or ActiveX™ controls or other active content technologies.

The analysis function can also be embodied in computer program products and used in the systems described above or other computer- or internet-based systems. Accordingly, another aspect of the present disclosure relates to a computer program product comprising a computer-usable medium having computer-readable program codes or instructions embodied thereon for enabling a processor to carry out gene mutation or expression analysis. These computer program instructions may be loaded onto a computer or other programmable apparatus to produce a machine, such that the instructions which execute on the computer or other programmable apparatus create means for implementing the functions or steps described above. These computer program instructions may also be stored in a computer-readable memory or medium that can direct a computer or other programmable apparatus to function in a particular manner, such that the instructions stored in the computer-readable memory or medium produce an article of manufacture including instruction means which implement the analysis. The computer program instructions may also be loaded onto a computer or other programmable apparatus to cause a series of operational steps to be performed on the computer or other programmable apparatus to produce a computer implemented process such that the instructions which execute on the computer or other programmable apparatus provide steps for implementing the functions or steps described above.

Thus in some embodiments the disclosure provides a method comprising: accessing information on a patient's APM gene status (e.g., presence or absence of mutations in an APM gene listed in Table 1 or Table 3, decreased or absent expression of a transcript or protein encoded by such gene) stored in a computer-readable tangible medium; querying this information to determine whether a sample obtained from the patient harbors a deficiency in at least one gene of a panel of genes comprising at least 1, 2, 3, 4, 5, 6, 7, 8, 9, 10, 11, 12, 13, 14, or 15 test genes selected from the genes listed in Table 1 or Table 3; outputting [or displaying] the quantitative or qualitative (e.g., “increased”) likelihood that the patient will respond (or be resistant) to a treatment regimen comprising an immune checkpoint inhibitor. As used herein in the context of computer-implemented embodiments of the disclosure, “displaying” means communicating any information by any sensory means. Examples include, but are not limited to, visual displays, e.g., on a computer screen or on a sheet of paper printed at the command of the computer, and auditory displays, e.g., computer generated or recorded auditory expression of a patient's genotype or expression.

The practice of the present disclosure may also employ other biology methods, software and systems. Computer software products of the disclosure typically include computer readable media having computer-executable instructions for performing the logic steps of the method of the disclosure. Suitable computer readable medium include floppy disk, CD-ROM/DVD/DVD-ROM, hard-disk drive, flash memory, ROM/RAM, magnetic tapes and etc. Basic computational biology methods are described in, for example, Setubal et al., INTRODUCTION TO COMPUTATIONAL BIOLOGY METHODS (PWS Publishing Company, Boston, 1997); Salzberg et al. (Ed.), COMPUTATIONAL METHODS IN MOLECULAR BIOLOGY, (Elsevier, Amsterdam, 1998); Rashidi & Buehler, BIOINFORMATICS BASICS: APPLICATION IN BIOLOGICAL SCIENCE AND MEDICINE (CRC Press, London, 2000); and Ouelette & Bzevanis, BIOINFORMATICS: A PRACTICAL GUIDE FOR ANALYSIS OF GENE AND PROTEINS (Wiley & Sons, Inc., 2^{nd} ed., 2001); see also, U.S. Pat. No. 6,420,108.

The present disclosure may also make use of various computer program products and software for a variety of purposes, such as probe design, management of data, analysis, and instrument operation. See U.S. Pat. Nos. 5,593,839; 5,795,716; 5,733,729; 5,974,164; 6,066,454; 6,090,555; 6,185,561; 6,188,783; 6,223,127; 6,229,911 and 6,308,170. Additionally, the present disclosure may have embodiments that include methods for providing genetic information over networks such as the Internet as shown in U.S. Ser. Nos. 10/197,621 (U.S. Pub. No. 20030097222); Ser. No. 10/063,559 (U.S. Pub. No. 20020183936), Ser. No. 10/065,856 (U.S. Pub. No. 20030100995); Ser. No. 10/065,868 (U.S. Pub. No. 20030120432); Ser. No. 10/423,403 (U.S. Pub. No. 20040049354).

Techniques for analyzing such expression, activity, and/or sequence data (indeed any data obtained according to the disclosure) will often be implemented using hardware, software or a combination thereof in one or more computer systems or other processing systems capable of effectuating such analysis.

### G. Kits

In another aspect of the present disclosure, a kit is described for practicing the methods or for use in the systems of the present disclosure. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit includes various components useful in detecting deficiency in one or more APM genes and, optionally, one or more housekeeping gene markers, using the above-discussed detection techniques. For example, the kit many include oligonucleotides specifically hybridizing under high stringency to DNA, mRNA or cDNA of one or more of the genes in Table 1 or Table 3. Such oligonucleotides can be used as PCR primers in RT-PCR reactions or hybridization probes. In some embodiments the kit comprises reagents (e.g., probes, primers, and or antibodies) for determining the sequence or expression level of a panel of genes, where said panel comprises at least 25%, 30%, 40%, 50%, 60%, 75%, 80%, 90%, 95%, 99%, or 100% genes from Table 1 or Table 3. In some embodiments the kit consists of reagents (e.g., probes, primers, and or antibodies) for determining the expression level of no more than 5, 10, 20, 30, 40, 50, 60, 70, 80, 90, 100, 125, 150, 175, 200, 250, 300, 250, 400, 450, 500, 550, 600, 650, 700, 750, 800, 850, 900, 1000, 1250, 1500, 1750, 2000, 2250, 2500, 2750, 3000, 3500, 4000, 4500, 5000, 6000, 7000, 8000, 9000, 10000, 12500, 15000, 17500, 20000 or more genes, wherein at least 1, 2, 3, 4, 5, 6, 7, 8, 9, 10, 11, 12, 13, 14, or 15 of these genes are genes from Table 1 or Table 3.

The oligonucleotides in the detection kit can be labeled with any suitable detection marker including but not limited to, radioactive isotopes, fluorophores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit may be unlabeled, and instead, one or more markers are provided in the kit so that users may label the oligonucleotides at the time of use.

In another embodiment of the disclosure, the detection kit contains one or more antibodies selectively immunoreactive with one or more proteins encoded by one or more gene listed in Table 1 or Table 3.

Various other components useful in the detection techniques may also be included in the detection kit of this disclosure. Examples of such components include, but are not limited to, Taq polymerase, deoxyribonucleotides, dideoxyribonucleotides, other primers suitable for the amplification of a target DNA sequence, RNase A, and the like. In addition, the detection kit preferably includes instructions on using the kit to practice the methods or utilize the systems of the present disclosure using human samples.

### H. Compositions Useful in the Preceding Aspects of the Disclosure

In one aspect, the disclosure provides compositions for use in the above methods, systems or kits. Such compositions include, but are not limited to, nucleic acid probes hybridizing to, an APM gene listed in Table 1 or Table 3 (or to any nucleic acids encoded thereby or complementary thereto); nucleic acid primers and primer pairs suitable for selectively amplifying all or a portion of the APM gene or any nucleic acids encoded thereby; antibodies binding immunologically to a polypeptide encoded by the APM gene; probe sets comprising a plurality of said nucleic acid probes, nucleic acid primers, antibodies, and/or polypeptides; microarrays comprising any of these; kits comprising any of these; etc.

The probe can generally be of any suitable size/length. In some embodiments the probe has a length from about 8 to 200, 15 to 150, 15 to 100, 15 to 75, 15 to 60, or 20 to 55 bases in length. They can be labeled with detectable markers with any suitable detection marker including but not limited to, radioactive isotopes, fluorophores, biotin, enzymes (e.g., alkaline phosphatase), enzyme substrates, ligands and antibodies, etc. See Jablonski et al., NUCLEIC ACIDS RES. (1986) 14:6115-6128; Nguyen et al., BIOTECHNIQUES (1992) 13:116-123; Rigby et al., J. MOL. BIOL. (1977) 113:237-251. Indeed, probes may be modified in any conventional manner for various molecular biological applications. Techniques for producing and using such oligonucleotide probes are conventional in the art.

Probes according to the disclosure can be used in the hybridization/amplification/detection techniques discussed above. Thus, some embodiments of the disclosure comprise probe sets suitable for use in a microarray in detecting, amplifying and/or quantitating a plurality of APM genes. In some embodiments the probe sets have a certain proportion of their probes directed to APM genes—e.g., a probe set consisting of 10%, 20%, 30%, 40%, 50%, 55%, 60%, 65%, 70%, 75%, 80%, 85%, 90%, 95%, 96%, 97%, 98%, or 99% probes specific for APM genes. In some embodiments the probe set comprises probes directed to at least 1, 2, 3, 4, 5, 6, 7, 8, 9, 10, 11, 12, 13, 14, or 15 of the genes in Table 1 or Table 3. Such probe sets can be incorporated into high-density arrays comprising 5,000, 10,000, 20,000, 50,000, 100,000, 200,000, 300,000, 400,000, 500,000, 600,000, 700,000, 800,000, 900,000, or 1,000,000 or more different probes. In other embodiments the probe sets comprise primers (e.g., primer pairs) for amplifying nucleic acids comprising at least a portion of one or more of the APM genes in Table 1 or Table 3.

### I. Embodiments of the Preceding Aspects of the Disclosure

Except where expressly stated, or where the context clearly suggests, otherwise, each of the following details represents a contemplated embodiment of each of the preceding aspects of the disclosure as if, for each embodiment, all details of the description above were reproduced below and vice versa.

In some embodiments, the immune checkpoint inhibitor is any of the therapeutic agents listed in Table 2 below. In some embodiments, the cancer is any of the example indications listed in Table 2 below.

TABLE 2

| Drug | | | Example | |
|---|---|---|---|---|
| (alternative name(s)) | Drug Developer | Target | indication(s) | Reference |
| Yervoy (ipilimumab, | Bristol-Myers | CTLA4 | Melanoma, NSCLC, | Gulley & Dahut, NAT. |
| MDX-010, MDX-101) | Squibb | | SCLC, bladder | CLIN. PRACTICE |
| | | | cancer, prostate | ONCOL. (2007) 4: |
| | | | cancer | 136-137 |
| Tremelimumab | AstraZeneca | CTLA4 | Mesothelioma | Ribas et al., |
| (ticilimumab, CP- | | | | ONCOLOGIST (2007) |
| 675, 206) | | | | 12: 873-883 |
| Opdivo (nivolumab) | Bristol-Myers | PD1 | Malignant | Brahmer et al., J. CLIN. |
| | Squibb | | melanoma | ONCOL. (2010) |
| | | | | 28: 3167-3175 |
| Keytruda | Merck & Co. | PD1 | Malignant | Hamid et al., N. ENGL. |
| (pembrolizumab, | | | melanoma | J. MED. (2013) |
| lambrolizumab, MK- | | | | 369: 134-144 |
| 3475) | | | | |
| MEDI4736 | AstraZeneca | PDL1 | NSCLC | Lee & Chow, TRANSL. |
| | | | | LUNG CANCER RES. |
| | | | | (2014) 3: 408-410 |
| MPDL3280A | Roche/Genentech | PDL1 | Urothelial bladder | Powles et al., NATURE |
| | | | cancer or NSCLC | (2014) 515: 558-562 |
| Pidilizumab (CT-011) | CureTech | PD1 | Hematologic or | Berger et al., CLIN. |
| | | | solid tumors | CANCER RES. (2008) |
| | | | | 14: 3044-3051 |
| lirilumab (BMS- | Bristol-Myers | KIR | Hematologic or | Kohrt et al., BLOOD |
| 986015) | Squibb | | solid tumors | (2014) 123: 678-686 |
| Indoximod (NLG- | Newlink Genetics | IDO1 | Breast cancer | Soliman et al., |
| 9189) | | | | ONCOTARGET (2014) |
| | | | | 5: 8136-8146 |
| INCB024360 | Incyte | IDO1 | Solid tumors | Koblish et al., MOL. |
| | | | | CANCER THER. (2010) |
| | | | | 9: 489-498 |
| MEDI0680 (AMP-514) | AstraZeneca | PD1 | Solid tumors | |
| MSB-0010718C | Merck KGaA | PDL1 | Solid tumors | |
| PF-05082566 | Pfizer | 4-1BB (also | Hematologic or | Fisher et al., CANCER |
| | | known as | solid tumors | IMMUNOL. IMMUNOTHER. |
| | | CD137) | | (2012) 61: 1721-33 |
| MEDI6469 | AstraZeneca | OX40 (also | Solid tumors | |
| | | known as | | |
| | | CD134) | | |
| BMS-986016 | Bristol-Myers | LAG3 | Hematologic or | |
| | Squibb | | solid tumors | |
| NLG-919 | Newlink Genetics | IDO1 | Solid tumors | |
| Urelumab (BMS- | Bristol-Myers | 4-1BB (also | Hematologic or | Li & Liu, CLIN. |
| 663513) | Squibb | known as | solid tumors | PHARMACOL. (2013) 5 |
| | | CD137) | | (Suppl. 1): 47-53 |

In some embodiments, the sequence of at least a portion of each test gene in a panel of genes can be determined by resequencing of the test genes. This can be done using a technique such as Sanger sequencing or massively-parallel sequencing of either targeted loci (e.g., hotspots) within the gene or effectively the entire gene. In this context, sequencing of effectively the entire gene can include sequencing of all exons (or all coding exons) optionally together with some portion (e.g., 5, 10, 20 or more nucleotides) of the intron upstream and/or downstream of each exon. Such an assay can include enrichment of genomic DNA of the sample for those fragments containing test genes to be analyzed (or containing fragments that collectively encompass all the regions of the tests genes to be analyzed) using kits designed for this purpose (e.g., Agilent SureSelect™, Illumina TruSeq Capture™, and Nimblegen SeqCap EZ Choice™). For example, genomic DNA containing the genes (or fragments thereof) to be analyzed can be hybridized to biotinylated capture RNA fragments to form biotinylated RNA (or DNA)/genomic DNA complexes. Alternatively, DNA capture probes may be utilized resulting in the formation of biotinylated DNA/genomic DNA hybrids. Other DNA capture probes (i.e., not utilizing a biotin and/or streptavidin system) can also be used to extract, enrich, or otherwise separate DNA of interest. Streptavidin coated magnetic beads and a magnetic force can be used to separate the biotinylated RNA (or DNA)/genomic DNA complexes from those genomic DNA fragments not present within a biotinylated RNA/genomic DNA complex. The obtained biotinylated RNA (or DNA)/genomic DNA complexes can be treated to remove the captured RNA (or DNA) from the magnetic beads, thereby leaving intact genomic DNA fragments containing a locus to be analyzed. Intact genomic DNA fragments containing the genes (or fragments thereof) to be analyzed (whether such fragments were extracted or otherwise enriched or separated using this biotin/streptavidin approach or some other technique) can be amplified using, for example, PCR techniques. The amplified genomic DNA fragments can be sequenced using a high-throughput sequencing technology or a next-generation sequencing technology such as Illumina HiSeg™, Illumina MiSeq™, Life Technologies SoLID™ or Ion Torrent™, or Roche 454™.

In some embodiments (i) the panel consists of no more than 5, 10, 20, 30, 40, 50, 60, 70, 80, 90, 100, 125, 150, 175, 200, 250, 300, 250, 400, 450, 500, 550, 600, 650, 700, 750, 800, 850, 900, 1000, 1250, 1500, 1750, 2000, 2250, 2500, 2750, 3000, 3500, 4000, 4500, 5000, 6000, 7000, 8000, 9000, 10000, 12500, 15000, 17500, 20000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

As discussed above, the methods of the disclosure generally involve determining the sequence of a panel of genes comprising APM genes. With modern high-throughput techniques, it is often possible to determine the sequence of tens, hundreds or thousands of genes. Indeed, it is possible to determine the sequence of the entire transcriptome (i.e., each transcribed sequence in the genome). Once such a global assay has been performed, one may then informatically analyze one or more subsets of genes (i.e., panels or pluralities of test genes). After sequencing of hundreds or thousands of genes in a sample, for example, one may analyze (e.g., informatically) the sequences of a panel or plurality of test genes comprising primarily genes selected from Table 1 or Table 3 according to the present disclosure.

A patient generally has an “increased likelihood” of some clinical feature or outcome (e.g., response or resistance) if the probability of the patient having the feature or outcome exceeds some reference probability or value. The reference probability may be the probability of the feature or outcome across the general relevant patient population. For example, if the probability (or likelihood) of response (or resistance) to a treatment regimen comprising an immune checkpoint inhibitor (e.g., a therapeutic agent listed in Table 2) in the relevant patient population (e.g., patients for whom such treatment is indicated, patients for whom such treatment is approved by a regulatory agency (e.g., the U.S. Food and Drug Administration), etc.) is X % and a particular (e.g., test) patient has been determined by the methods of the present disclosure to have a probability (or likelihood) of response (or resistance) of Y %, and if Y>X, then in some embodiments the patient has an “increased likelihood” of response. In another example, if the probability of cancer recurrence after surgery in the general breast cancer patient population (or some specific subpopulation) is X % and a particular patient has been determined by the methods of the present disclosure to have a probability of recurrence of Y %, and if Y>X, then in some embodiments the patient has an “increased likelihood” of response. In some embodiments the test patient is determined to have an increased likelihood of response to treatment (e.g., treatment comprising an immune checkpoint inhibitor) if the test likelihood exceeds the reference likelihood by at least some threshold amount (e.g., at least 0.5, 0.75, 0.85, 0.90, 0.95, 1, 2, 3, 4, 5, 6, 7, 8, 9, or 10 or more fold or standard deviations or at least 1%, 2%, 3%, 4%, 5%, 10%, 15%, 20%, 25%, 30%, 35%, 40%, 45%, 50%, 55%, 60%, 65%, 75%, 80%, 85%, 90%, 95%, 96%, 97%, 98%, 99% or 100% or more greater than the reference likelihood).

A threshold or reference value (e.g., reference probability or likelihood) may be determined and a particular patient's probability of response may be compared to that threshold or reference. Such an index probability may represent the average probability (or likelihood) of the clinical feature in a set of individuals from a diverse cancer population or a subset of the population. For example, one may in some embodiments determine the likelihood of resistance to therapy comprising an immune checkpoint inhibitor in a random sampling of patients with some specific cancer (e.g., melanoma). This average likelihood may be termed the “threshold index likelihood”.

The results of any analyses according to the disclosure will often be communicated to physicians (or other healthcare providers) and/or patients (or other interested parties such as researchers) in a transmittable form that can be communicated or transmitted to any of the above parties. Such a form can vary and can be tangible or intangible. The results can be embodied in descriptive statements, diagrams, photographs, charts, images or any other visual forms. For example, graphs showing expression or activity level or sequence variation information for various genes can be used in explaining the results. Diagrams showing such information for additional target gene(s) are also useful in indicating some testing results. The statements and visual forms can be recorded on a tangible medium such as papers, computer readable media such as floppy disks, compact disks, etc., or on an intangible medium, e.g., an electronic medium in the form of email or website on internet or intranet. In addition, results can also be recorded in a sound form and transmitted through any suitable medium, e.g., analog or digital cable lines, fiber optic cables, etc., via telephone, facsimile, wireless mobile phone, internet phone and the like.

Thus, the information and data on a test result can be produced anywhere in the world and transmitted to a different location. As an illustrative example, when an expression level or sequencing (or genotyping) assay is conducted outside the United States, the information and data on a test result may be generated, cast in a transmittable form as described above, and then imported into the United States. Accordingly, the present disclosure also encompasses a method for producing a transmittable form of information on at least one of (a) expression level or (b) mutation status for at least one patient sample. The method comprises the steps of (1) determining at least one of (a) or (b) above according to methods of the present disclosure; and (2) embodying the result of the determining step in a transmittable form (e.g., recording the result in a tangible medium). The transmittable form can in some embodiments be a “product” of such a method.

### I. Additional Embodiments of the Disclosure

In some embodiments, the cancer is chosen from the group consisting of head & neck cancer (e.g., squamous cell carcinoma), brain cancer (e.g., glioblastoma), breast cancer (e.g., invasive ductal carcinoma, invasive lobular carcinoma), colorectal cancer (e.g., colon adenocarcinoma), lung cancer (e.g., adenosquamous, carcinoma, adenocarcinoma, large cell carcinoma, large cell neuroendocrine carcinoma, squamous cell carcinoma, non-small cell lung cancer, small cell lung cancer), ovarian cancer (e.g., epithelial), gastric cancer, melanoma, and prostate cancer. In some embodiments, the cancer is chosen from the group consisting of gastric cancer, endometrial cancer and colon cancer. In some embodiments, the cancer is chosen from the group consisting of lung cancer and melanoma.

In some embodiments, the panel of test genes comprises at least 1, 2, 3, 4, 5, 6, 7, 8, 9, 10, 11, 12, 13, 14, or 15 of the genes listed in Table 1. In some embodiments, the panel of test genes comprises at least 1, 2, 3, 4, 5, 6, 7, 8, 9, 10, 11, 12, 13, 14, or 15 of the [*] genes listed in Table 1 (i.e., genes indicated with an asterisk [*] in Table 1). In some embodiments, the panel of test genes comprises at least 1, 2, 3, or 4 of the genes listed in Table 3 below.

TABLE 3

| | | NCBI | | Chromosomal Location | | |
|---|---|---|---|---|---|---|
| Gene | Gene | Gene ID | | (NCBI Homo sapiens | | |
| # | Name | # | Description | Annotation Release 108) | Aliases | MIM |
| 1 | HLA-F | ID:3134 | major | Chromosome 6, | CDA12, H LA-5.4, | 143110 |
| | | | histocompatibility | NC_000006.12 | HLA-CDA12, | |
| | | | complex, class I, F | (29723340 . . . 29740355) | HLAF | |
| 2 | CUTA | ID:4261 | class II major | Chromosome 16, | C2TAIV, | 600005 |
| | | | histocompatibility | NC_000016.10 | MHC2TA, | |
| | | | complex | (10866208 . . . 10941562) | NLRA, CIITA | |
| | | | transactivator | | | |
| 3 | ERAP2 | ID:64167 | endoplasmic | Chromosome 5, | L-RAP, LRAP | 609497 |
| | | | reticulum | NC_000005.10 | | |
| | | | aminopeptidase 2 | (96875940 . . . 96919703) | | |
| 4 | PDIA3 | ID:2923 | protein disulfide | Chromosome 15, | ER60, ERp57, | 602046 |
| | | | isomerase family | NC_000015.10 | ERp60, ERp61, | |
| | | | A member 3 | (43746392 . . . 43772606) | GRP57, GRP58, | |
| | | | | | HEL-S-269, HEL- | |
| | | | | | S-93n, HsT17083, | |
| | | | | | P58, PI-PLC | |

In some embodiments, assaying test genes comprises (or the sample analyzer is configured to perform one or more assays comprising) one or more of the following: (a) extracting genomic DNA from a tumor sample (e.g., an FFPE sample); (b) enriching the resultant sample for DNA from the test genes; and (c) sequencing the enriched DNA to determine the sequence of all or a portion of each test gene. In some embodiments, assaying test genes comprises (or the sample analyzer is configured to perform one or more assays comprising) one or more of the following: (a) extracting RNA from a tumor sample (e.g., an FFPE sample); (b) enriching the resultant sample for RNA (or cDNA) from the test genes; and (c) measuring the level amount of the enriched RNA (or cDNA).

In some embodiments, DNA enrichment is achieved by contacting a sample with DNA hybridization capture probes having sequences at least partially complementary with one or more target sequences in the test genes and, e.g., washing away unbound DNA to leave only or substantially only DNA from the test genes in the resultant sample. In some embodiments, DNA enrichment is achieved by contacting a sample with PCR primers (and other PCR reagents, e.g., polymerase, nucleotides, etc.) having sequences at least partially complementary with one or more target sequences in the test genes and performing an amplification reaction to leave substantially only DNA from the test genes in the resultant sample. In some embodiments, DNA enrichment is achieved by a combination of such capture and amplification.

In some embodiments, DNA is fragmented (e.g., before enrichment). In some embodiments, sample-specific barcodes are attached to DNA to be sequenced (e.g., via A-tailed ligation of barcoded Illumina sequencing adaptors). In some embodiments, samples from multiple patients are pooled for hybridization capture. In some embodiments the capture pool comprises (or consists essentially of or consists of) hybridization probes collectively complementary to at least one known exon of each test gene. In some embodiments the capture pool comprises (or consists essentially of or consists of) hybridization probes collectively complementary to at least 10%, 20%, 30%, 40%, 50%, 60%, 70%, 80%, 90%, or 100% of known exons (or coding exons) of each test gene.

In some embodiments a detected variant is classified as polymorphism or potentially deleterious based on the effect on protein function (frame shifts, nonsense codons, splice donor or acceptor mutations), comparison to public databases (dbSNP, exome variant server, ExAC) and/or literature on known variants and functional assays.

In some embodiments, the method (or system) screens for any mutation in the test genes and the mutation(s) detected is(are) at least one of the specific mutations listed in Table 4. In some embodiments, the method (or system) assays for one or more pre-determined mutations selected from the specific mutations listed in Table 4.

TABLE 4

| Gene | Exon | Variant | HGVS |
|---|---|---|---|
| B2M | 1 | Base 43 del2 | c.43_44del (p.Leu15Phefs*41) |
| B2M | 2 | Base 209 del1 | c.276del (p.Thr93Leufs*10) |
| B2M | 2 | Base −2 A > G | c.68-2A > G |
| CIITA | 11 | Base 956 insC | c.1962dupC (p.Gly655Argfs*94), c.1965dupC |
| | | | (p.Gly656Argfs*94) |
| ERAP1 | 12 | Base 102 del4 | c.1861_1864del (p.Thr621Alafs*3) |
| ERAP2 | 3 | Base 91 del1 | c.805del (p.Cys269Valfs*6) |
| ERAP2 | 8 | Base 66 | c.1437_1438delinsAT (p.Ile480*) |
| | | del2insAT | |
| NLRC5 | 4 | Base 643 del4 | c.1067_1070del (p.Pro356Glnfs*20) |
| NLRC5 | 4 | Base 782 del1 | c.1206del (p.Cys403Valfs*33) |
| NLRC5 | 4 | Base 1221 C > T | c.1645C > T (p.Gln549*) |
| NLRC5 | 4 | Base 1350 C > T | c.1774C > T (p.Gln592*) |
| NLRC5 | 11 | Base 23 del1 | c.2566del (p.Val856Serfs*8) |
| NLRC5 | 23 | Base 78 del1 | c.3584del (p.Lys1195Argfs*38) |
| NLRC5 | 36 | Base 23 G > T | c.4606G > T (p.Glu1536*) |
| NLRC5 | 42 | Base 68 del1 | c.5149del (p.His1717Ilefs*29) |
| TAPBP | 4 | Base 92 del1 | c.561del (p.Thr188Profs*17), c.300del (p.Thr101Profs*17) |
| B2M | 1 | Base 3 G > C | c.3G > C (p.Met1?) |
| B2M | 1 | Base 69 T > C | c.67+2T > C |
| B2M | 2 | Base 148 del12 | c.215_226del (p.Ser72_Ser75del) |
| B2M | 2 | Base 235 G > T | c.302G > T (p.Arg101Leu) |
| B2M | 2 | Base 250 de134 | c.317_346+4del |
| CIITA | 11 | Base 132 C > T | c.1138C > T (p.Arg380Trp), c.1141C > T (p.Arg381Trp) |
| CIITA | 11 | Base 322 C > G | c.1328C > G (p.Pro443Arg), c.1331C > G (p.Pro444Arg) |
| ERAP2 | 6 | Base 107 T > G | c.1232T > G (p.Leu411Arg) |
| ERAP2 | 14 | Base 82 C > T | c.2251C > T (p.Arg751Cys) |
| NLRC5 | 17 | Base 69 C > A | c.3098C > A (p.Ala1033Asp) |
| NLRC5 | 22 | Base 56 C > T | c.3478C > T (p.Pro1160Ser) |
| NLRC5 | 36 | Base 30 G > A | c.4613G > A (p.Gly1538Asp) |
| NLRC5 | 47 | Base −1 G > A | c.5490−1G > A |
| TAP1 | 7 | Base 170 C > T | c.1727C > T (p.Pro576Leu), c.944C > T (p.Pro315Leu) |
| TAP1 | 7 | Base 188 A > C | c.1745A > C (p.Gln582Pro), c.962A > C (p.Gln321Pro) |
| TAP1 | 10 | Base 40 G > A | c.2123G > A (p.Arg708Gln), c.1340G > A (p.Arg447Gln) |
| TAP2 | 3 | Base −1 G > A | c.609−1G > A |
| TAP2 | 4 | Base 199 G > A | c.938G > A (p.Arg313His) |
| TAP2 | 7 | Base 127 del1 | c.1399del (p.Val467Leufs*2) |
| TAPBP | 2 | Base 66 G > A | c.103G > A (p.Gly35Arg) |
| TAPBP | 2 | Base 124 C > G | c.161C > G (p.Pro54Arg) |
| TAPBP | 2 | Base 137 | c.174_175delinsTT (p.Asp59delinsTyr) |
| | | del2insTT | |
| TAPBP | 4 | Base 261 | c.730_731delinsAT (p.Asp244delinsIle), c.469_470delinsAT |
| | | del2insAT | (p.Asp157delinsIle) |

In some embodiments the cancer is breast cancer (e.g., invasive ductal breast carcinoma, invasive lobular breast cancer, etc.) and the panel comprises one or more of the transporter associated with antigen processing genes listed in Table 1 (e.g., TAP1, TAP2, TABP). In some embodiments the cancer is colon or colorectal cancer (e.g., colon adenocarcinoma) and the panel comprises one or both of B2M and NLRC5.

In some embodiments the cancer is colorectal cancer and the method comprises (or the system is configured perform analysis comprising) microsatellite stability analysis. In some embodiments the cancer is known to be microsatellite unstable. In some embodiments the cancer is known to be microsatellite stable.

### Specific Embodiments

The following paragraphs describe numerous, but non-limiting, specific embodiments of the present disclosure.

Embodiment 1. A method for detecting mutations in a panel of genes in a sample from a patient identified as having cancer, the method comprising:
(1) obtaining, or providing, one or more samples from a patient identified as having cancer; and
(2) assaying the sample to determine the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3;
wherein (a) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (b) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

Embodiment 2. A method for measuring expression in a panel of genes in a sample from a patient identified as having cancer, the method comprising:
(1) obtaining, or providing, one or more samples from a patient identified as having cancer; and
(2) assaying the sample to determine the expression of each test gene in a panel of genes comprising at least 1, 2, 3, 4, 5, 6, 7, 8, 9, 10, 11, 12, 13, 14, or 15 test genes selected from the genes listed in Table 1 or Table 3;
wherein (a) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (b) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

Embodiment 3. A method for treating cancer patients comprising:
(1) assaying one or more patient samples comprising or derived from a cancer cell to determine the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3;
(2) determining whether any of the test genes harbors a mutation; and
(3)(a) recommending, prescribing or administering a treatment regimen comprising an immune checkpoint inhibitor to a patient in whose sample no test gene is determined in (2) to have a mutation or
(3)(b) recommending, prescribing or administering a treatment regimen not comprising an immune checkpoint inhibitor to a patient in whose sample at least one test gene is determined in (2) to have a mutation.

Embodiment 4. A method for detecting resistance (and/or an increased likelihood of resistance) to a treatment regimen comprising an immune checkpoint inhibitor, the method comprising:
(1) assaying one or more patient samples comprising or derived from a cancer cell to determine the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3;
(2) determining whether any of the test genes harbors a mutation; and
(3)(a) recording in a tangible medium that a patient in whose sample at least one test gene is determined in (2) to have a mutation has an increased likelihood of resistance to a treatment regimen comprising an immune checkpoint inhibitor or
(3)(b) recording in a tangible medium that a patient in whose sample no test gene is determined in (2) to have a mutation has a decreased likelihood of resistance to a treatment regimen comprising an immune checkpoint inhibitor.

Embodiment 5. A method for treating cancer patients comprising:
(1) assaying one or more patient samples comprising or derived from a cancer cell to measure expression of a panel of genes comprising at least 1, 2, 3, 4, 5, 6, 7, 8, 9, 10, 11, 12, 13, 14, or 15 test genes selected from the genes listed in Table 1 or Table 3;
(2) determining whether any of the test genes or any protein encoded thereby has low (including undetectable) expression in the sample(s); and
(3)(a) recommending, prescribing or administering a treatment regimen comprising an immune checkpoint inhibitor to a patient in whose sample no test gene is determined in (2) to have low expression or
(3)(b) recommending, prescribing or administering a treatment regimen not comprising an immune checkpoint inhibitor to a patient in whose sample at least one test gene is determined in (2) to have low expression.

Embodiment 6. A method for detecting resistance (and/or an increased likelihood of resistance) to a treatment regimen comprising an immune checkpoint inhibitor, the method comprising:
(1) assaying one or more patient samples comprising or derived from a cancer cell to measure expression of a panel of genes comprising at least 1, 2, 3, 4, 5, 6, 7, 8, 9, 10, 11, 12, 13, 14, or 15 test genes selected from the genes listed in Table 1 or Table 3;
(2) determining any of the test genes or any protein encoded thereby has low (including undetectable) expression in the sample(s); and
(3)(a) recording in a tangible medium that a patient in whose sample at least one test gene is determined in (2) to have low expression has an increased likelihood of resistance to a treatment regimen comprising an immune checkpoint inhibitor or
(3)(b) recording in a tangible medium that a patient in whose sample no test gene is determined in (2) to have low expression has a decreased likelihood of resistance to a treatment regimen comprising an immune checkpoint inhibitor.

Embodiment 7. A system for detecting resistance (and/or an increased likelihood of resistance) to a treatment regimen comprising an immune checkpoint inhibitor, the system comprising:
(1) a sample analyzer for assaying one or more patient samples comprising or derived from a cancer cell to determine the sequence of at least a portion of each test gene in a panel of genes comprising at least 1, 2, 3, 4, 5, 6, 7, 8, 9, 10, 11, 12, 13, 14, or 15 test genes selected from the genes listed in Table 1 or Table 3, wherein the sample analyzer contains the sample or DNA molecules extracted or derived from the sample;
(2) a first computer program for receiving test gene sequence data on the test genes;
(3) a second computer program for comparing the test gene sequence data to one or more reference gene sequences for each test gene to determine whether any of the test genes harbors a mutation; and
(4) a third computer program for determining
   (a) that a patient in whose sample at least one test gene is determined by the second computer program in (3) to have a mutation has an increased likelihood of resistance to a treatment regimen comprising an immune checkpoint inhibitor or
   (b) that a patient in whose sample no test gene is determined by the second computer program in (2) to have a mutation has a decreased likelihood of resistance to a treatment regimen comprising an immune checkpoint inhibitor. In some embodiments, the system further comprises a display module displaying the comparison between the test sequence(s) and the reference sequence(s), or displaying a result of the computerized comparison.

Embodiment 8. A system for detecting resistance (and/or an increased likelihood of resistance) to a treatment regimen comprising an immune checkpoint inhibitor, the system comprising:
(1) a sample analyzer for assaying one or more patient samples comprising or derived from a cancer cell to measure expression of a panel of genes comprising at least 1, 2, 3, 4, 5, 6, 7, 8, 9, 10, 11, 12, 13, 14, or 15 test genes selected from the genes listed in Table 1 or Table 3, wherein the sample analyzer contains the sample or DNA, RNA or protein molecules extracted or derived from the sample;
(2) a first computer program for receiving test expression data on the test genes;
(3) a second computer program for comparing the test expression data to one or more reference expression values for each test gene to determine whether any of the test genes has low (including undetectable) expression; and
(4) a third computer program for determining
   (a) that a patient in whose sample at least one test gene is determined by the second computer program in (3) to have low expression has an increased likelihood of resistance to a treatment regimen comprising an immune checkpoint inhibitor or
   (b) that a patient in whose sample no test gene is determined by the second computer program in (2) to have low expression has a decreased likelihood of resistance to a treatment regimen comprising an immune checkpoint inhibitor.

Embodiment 9. A diagnostic kit for detecting resistance (and/or an increased likelihood of resistance) to a treatment regimen comprising an immune checkpoint inhibitor, the kit comprising, in a compartmentalized container, a plurality of oligonucleotides hybridizing to a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

Embodiment 10. A kit consisting essentially of, in a compartmentalized container, a plurality of PCR reaction mixtures for PCR amplification of DNA from a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes, and wherein each reaction mixture comprises a PCR primer pair for PCR amplifying DNA that corresponds to one of the test genes.

Embodiment 11. The kit of either Embodiment 9 or 10, wherein the oligonucleotides are hybridizing probes for hybridization with an amplification product of the test gene(s) (e.g., an amplification product of DNA corresponding to the gene) under stringent conditions or primers suitable for PCR amplification of the test genes (e.g., suitable for amplification of DNA of a sample obtained from a tumor sample).

Embodiment 12. The kit of any one of Embodiments 9-11, wherein the probes and/or the primers are labelled (e.g., with a fluorescent tag).

Embodiment 13. The kit of any one of Embodiments 9-12, further comprising instructions for detecting resistance (and/or an increased likelihood of resistance) to a treatment regimen comprising an immune checkpoint inhibitor based at least in part on the presence or absence of mutations in the test genes.

Embodiment 14. The kit of any one of Embodiments 9-13, further comprising one or more computer software programs for detecting resistance (and/or an increased likelihood of resistance) to a treatment regimen comprising an immune checkpoint inhibitor based at least in part on the presence or absence of mutations in the test genes.

Embodiment 15. The kit of Embodiment 14, wherein the computer software program is capable of communicating (e.g., display) or instructing a computer to record in a tangible medium whether (a) a patient in whose sample at least one test gene is determined in (2) to have a mutation has an increased likelihood of resistance to a treatment regimen comprising an immune checkpoint inhibitor or (b) a patient in whose sample no test gene is determined in (2) to have a mutation has a decreased likelihood of resistance to a treatment regimen comprising an immune checkpoint inhibitor.

Embodiment 16. Use of a plurality of oligonucleotides for hybridization under stringent conditions to, or primers suitable for PCR amplification of DNA that corresponds to a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes; for determining the sequence of the test genes in a sample from a patient having cancer, for detecting resistance (and/or an increased likelihood of resistance) to a treatment regimen comprising an immune checkpoint inhibitor, wherein (a) the presence of a mutation in at least one test gene indicates an increased likelihood of resistance and (b) no detected mutation in any test gene indicates no increased likelihood of resistance.

Embodiment 17. Use of a plurality of oligonucleotides for hybridization under stringent conditions to, or primers suitable for PCR amplification of DNA that corresponds to a panel of genes comprising at least 1, 2, 3, 4, 5, 6, 7, 8, 9, 10, 11, 12, 13, 14, or 15 test genes selected from the genes listed in Table 1 or Table 3, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes; for measuring expression of the test genes in a sample from a patient having cancer, for detecting resistance (and/or an increased likelihood of resistance) to a treatment regimen comprising an immune checkpoint inhibitor, wherein (a) low (including undetectable) expression in at least one test gene indicates an increased likelihood of resistance and (b) absence of decreased expression in any test gene indicates no increased likelihood of resistance.

Embodiment 18. The use of either Embodiment 16 or 17, wherein the probes and/or the primers are labelled (e.g., with a fluorescent tag).

Embodiment 19. The method of any one of Embodiments 1-6, the system of either Embodiment 7 or 8, the kit of any one of Embodiments 9-15, or the use of any one of Embodiments 16-18, wherein the cancer is melanoma, renal cancer, lung cancer (e.g., NSCLC, SCLC, mesothelioma), bladder cancer (e.g., urothelial bladder cancer), breast cancer, gastric cancer, prostate cancer, HNSCC, or hematologic cancer.

Embodiment 20. The method of any one of Embodiments 1-6, the system of either Embodiment 7 or 8, the kit of any one of Embodiments 9-15, or the use of any one of Embodiments 16-18, wherein the immune checkpoint inhibitor is any of the agents listed in Table 2.

Embodiment 21. The method of any one of Embodiments 3-6 or the system of either Embodiment 7 or 8, wherein (i) the panel consists of no more than 20, 30, 40, 50, 60, 70, 80, 90, 100, 125, 150, 175, 200, 250, 300, 250, 400, 450, 500, 550, 600, 650, 700, 750, 800, 850, 900, 1000 or more genes, and/or (ii) the test genes represent at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 60%, 70%, 75%, 80%, 85%, 90%, 95%, 96%, 97%, 98%, 99% or 100% of the panel of genes.

### EXAMPLES

### Example 1

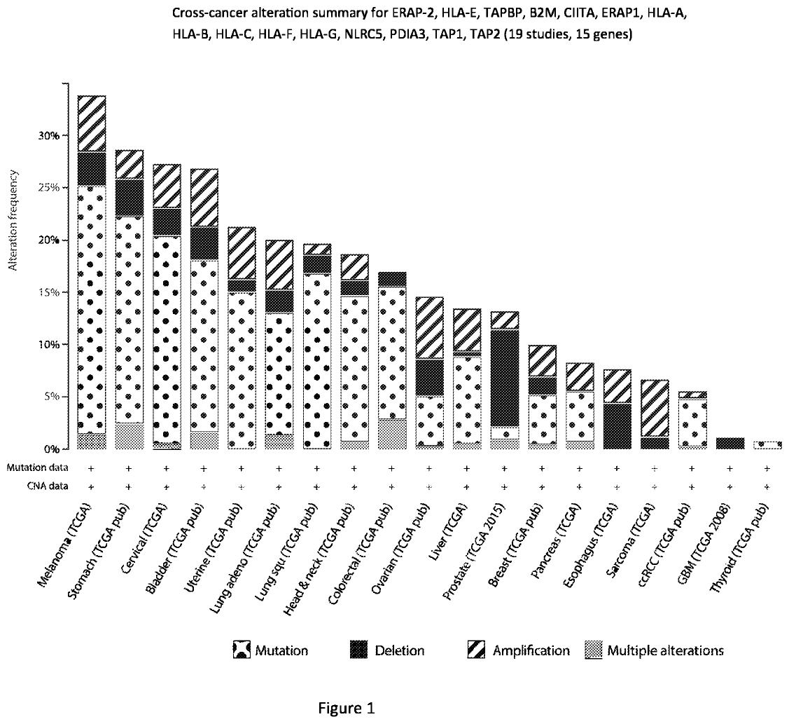

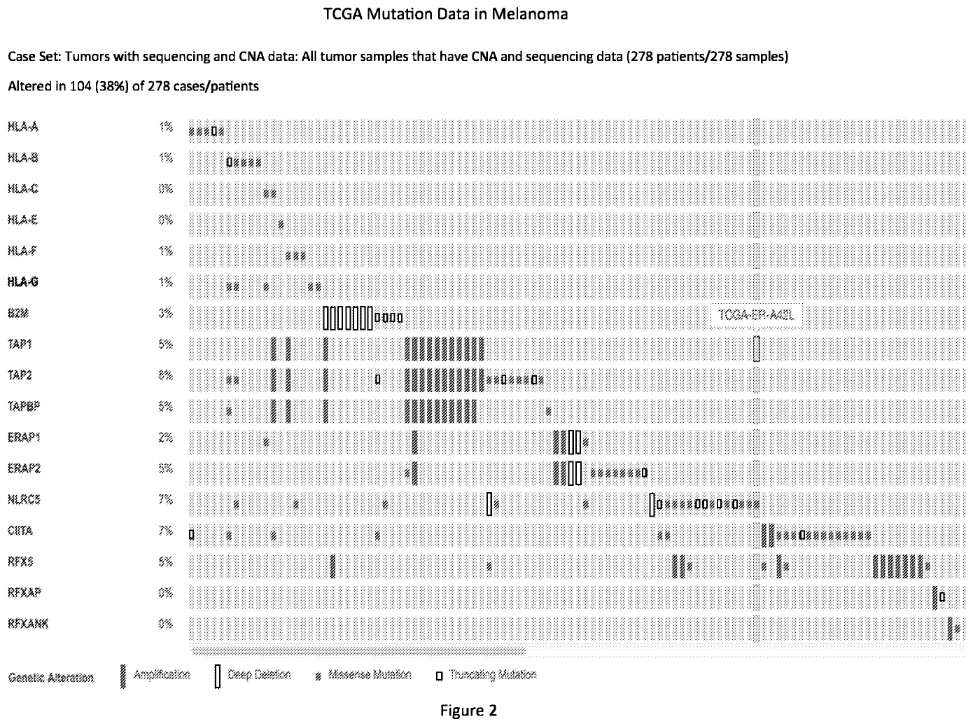

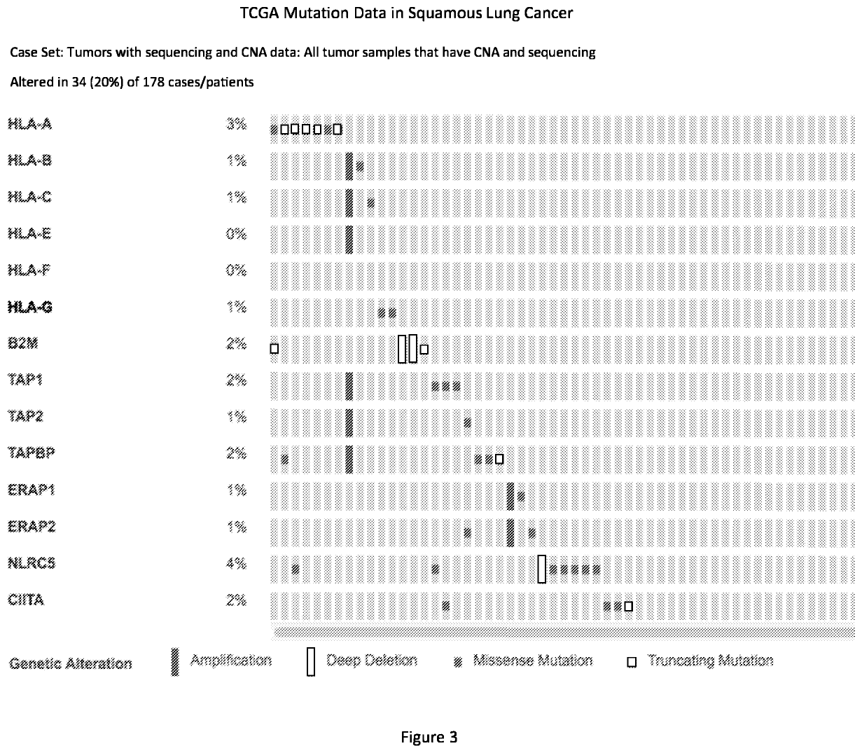

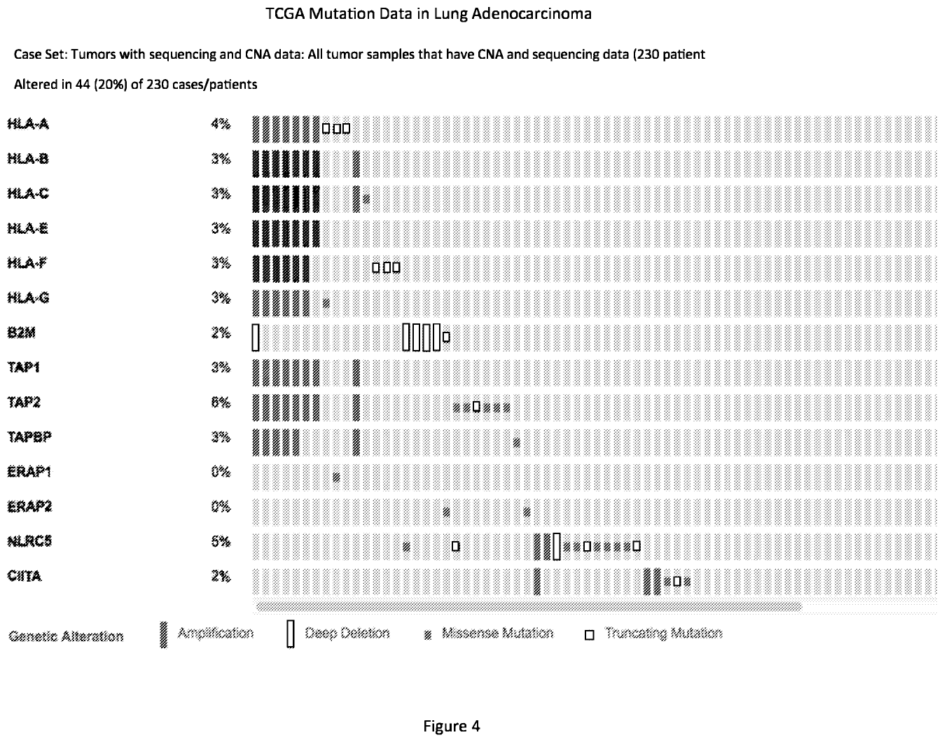

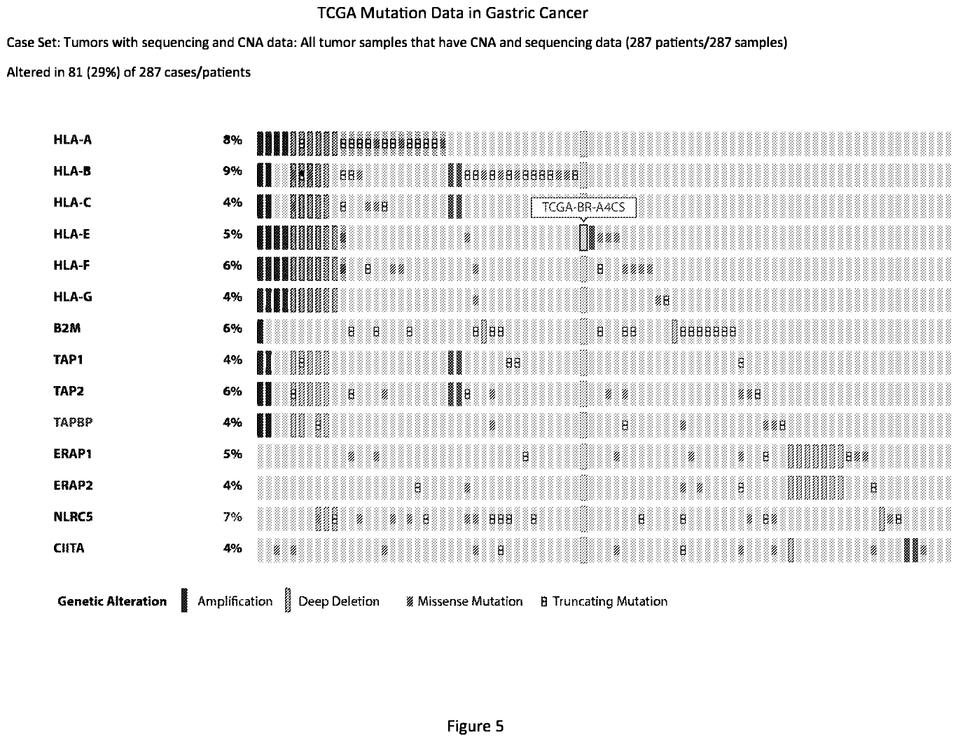

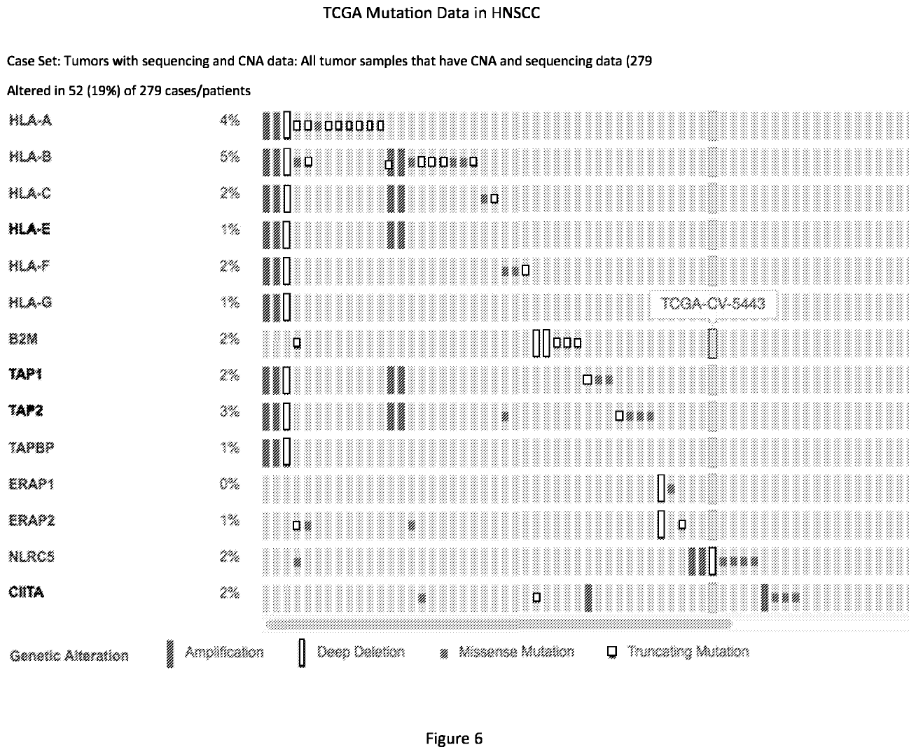

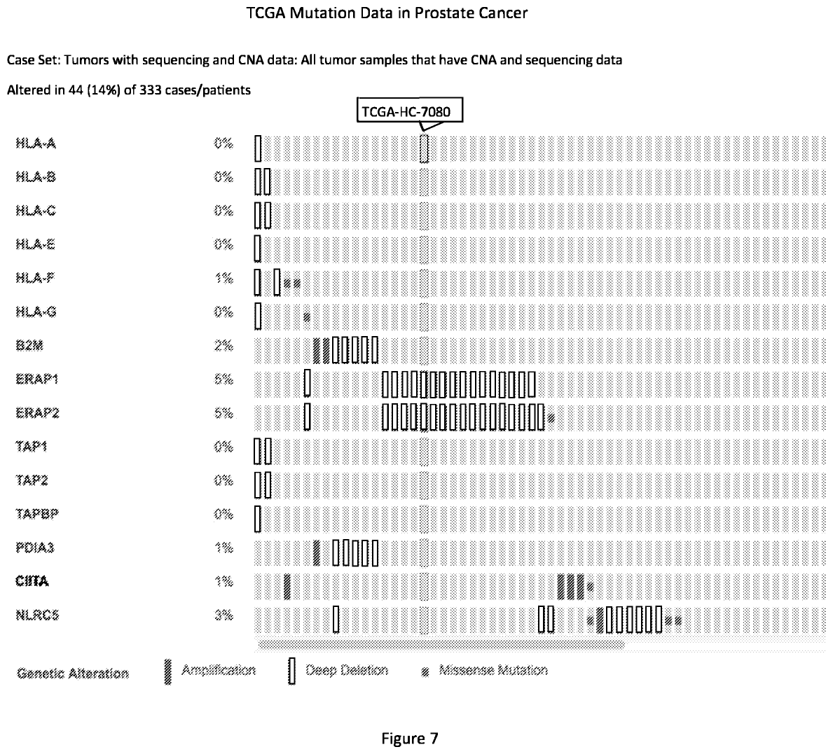

Using mutation and copy number data available through The Cancer Genome Atlas consortium website a panel of antigen processing machinery (APM) genes (see Table 1 above) was examined for copy number loss or mutations in various cancers. Several tumor types showed mutation rates or deletions in many or all of the APM genes in a largely mutually exclusive manner (see FIGS. 1-7). This suggests that loss of one of the APM components is sufficient for a tumor to achieve immune escape.

The presence and frequency of inactivating mutations in APM genes is consistent with a process driven by increased immunogenicity. Tumors with a high frequency of repair defects (e.g., MSI positive) such as gastric cancer, endometrial cancer and colon cancer show frequent inactivation of APM components.

TABLE A
Percent tumors with putative APM deficiency by tumor type

| | | # of tumors with |
|---|---|---|
| | | deletion or mutation |
| Cancer Type | N | in an APM gene (%) |
| Head & Neck SCC | 279 | 37 (13%) |
| Lung Squamous Carcinoma | 178 | 32 (18%) |
| Lung Adenocarcinoma | 230 | 34 (15%) |
| Stomach Cancer | 230 | 71 (31%) |
| Melanoma | 278 | 77 (28%) |
| Prostate Cancer | 333 | 39 (12%) |

The high rate of neo-antigens generated by MSI may create a need for an effective immune escape mechanism in these cancers. Similarly, melanoma and lung cancer, highly immunogenic due to the mutagenic effect of exposure to UV and tobacco smoke, respectively, are also often defective in APM.

### Example 2

In order to apply the findings of Example 1 toward the development of a signature for detecting resistance to immune checkpoint inhibitors, we performed a detailed meta-analysis of data presented in Snyder et al., N. ENGL. J. MED. (2014) 371:2189-2199. There, tumor DNA from 64 malignant melanoma patients treated with the immune checkpoint inhibitor ipilimumab were exome sequenced for an entirely different purpose, i.e., in an attempt to correlated mutation load and drug response and to identify tumor epitopes. We searched published exome data from Snyder et al. for non-synonymous variations in APM genes. Clinically durable response (DCB) had been defined in Snyder et al. as radiographic evidence of freedom from disease or evidence of a stable or decreased volume disease for more than 6 month. By these criteria, 37 patients had durable response with a response duration mean of 149 weeks. The mean duration of response for patients with no clinical durable benefit (NCB) was 13 weeks. The results of this analysis are summarized in the following tables.

TABLE B

| | | Mean Duration | Median Duration |
|---|---|---|---|
| Group | N | Response (wks) | Response (wks) |
| All patients | 64 | 92 | 52 |
| DCB | 37 | 149 | 109 |
| NCB | 25 | 13 | 12 |
| APM deficient | 4 | 9 | 9 |
| APM competent | 60 | 98 | 62 |

TABLE C

| | APM | APM |
|---|---|---|
| Group | competent | deficient |
| NCB | 21 | 4 |
| DCB | 37 | 0 |

Four patients had mutations in APM genes (APM-deficient), one each in CIITA, ERAP2, HLA-F and PDIA3. The mean duration of response of APM mutation carriers was 9 weeks. All four APM mutant tumors were among those without clinical benefit. This shows that a panel of APM genes can be used by mutation screening to identify patients likely to be resistant to immune checkpoint inhibitors (e.g., ipilimumab).

### Example 3

The prevalence of mutations in APM genes was assessed by next-generation sequencing of a set of commercial tumor samples of various tissue origins. Tissue origins and tumor subtypes are summarized in Table D.

TABLE D

| Tissue | Subtype | Sample N |
|---|---|---|
| brain | GBM | 9 |
| breast | IDC | 144 |
| breast | ILC | 11 |
| colorectal | colonADC | 64 |
| endometrial | EC | 14 |
| lung | ADSQ | 2 |
| lung | CARC | 20 |
| lung | LCC | 7 |
| lung | LCNEC | 11 |
| lung | lungADC | 73 |
| lung | lungSCC | 79 |
| lung | NSCLC | 26 |
| lung | SCLC | 8 |
| ovary | OC | 15 |

| | |
|---|---|
| Total | 483 |

Each tumor sample had genomic DNA extracted from FFPE sections by standard methods. DNA was fragmented and sample-specific barcodes were attached via A-tailed ligation of barcoded Illumina sequencing adaptors. 16 samples were pooled for each hybridization capture. The capture pool consisted of exon-based hybridization probes for all known exons in 15 genes from the antigen-processing pathway (Table 1). Captured sequence fragments were amplified and sequenced on an Illlumina MiSeq instrument.

Mutation screening for non-HLA genes (* genes in Table 1) was performed using an informatic review application (HLA sequence data were not reviewed in this Example 3). Classification of variants as polymorphism or potentially deleterious was based on their effect on protein function (frame shifts, nonsense codons, splice donor or acceptor mutations), comparison to public databases (dbSNP, exome variant server, ExAC) and literature on known variants and functional assays.

Sequencing of 483 tumor samples identified 108 mutations (sequence variants classified as deleterious or suspected deleterious) in 86 samples. Table E lists the individual mutations and number of their observations. Frequencies by gene are tabulated in Table F.

TABLE E

| Gene | Exon | Variant | HGVS | Classification | Occurrences | |
|---|---|---|---|---|---|---|
| B2M | 1 | Base 43 del2 | c.43_44del | Deleterious | 3 | |
| | | | (p.Leu15Phefs*41) | | | |
| B2M | 2 | Base 209 del1 | c.276del | Deleterious | 1 | |
| | | | (p.Thr93Leufs*10) | | | |
| B2M | 2 | Base −2 A > G | c.68−2A > G | Deleterious | 2 | |
| CIITA | 11 | Base 956 insC | c.1962dupC | Deleterious | 1 | |
| | | | (p.Gly655Argfs*94), | | | |
| | | | c.1965dupC | | | |
| | | | (p.Gly656Argfs*94) | | | |
| ERAP1 | 12 | Base 102 del4 | c.1861_1864del | Deleterious | 1 | |
| | | | (p.Thr621Alafs*3) | | | |
| ERAP2 | 3 | Base 91 del1 | c.805del | Deleterious | 1 | |
| | | | (p.Cys269Valfs*6) | | | |
| ERAP2 | 8 | Base 66 | c.1437_1438delinsAT | Deleterious | 1 | |
| | | del2insAT | (p.Ile480*) | | | |
| NLRC5 | 4 | Base 643 del4 | c.1067_1070del | Deleterious | 1 | |
| | | | (p.Pro356Glnfs*20) | | | |
| NLRC5 | 4 | Base 782 del1 | c.1206del | Deleterious | 1 | |
| | | | (p.Cys403Valfs*33) | | | |
| NLRC5 | 4 | Base 1221 C > T | c.1645C > T (p.Gln549*) | Deleterious | 1 | |
| NLRC5 | 4 | Base 1350 C > T | c.1774C > T (p.Gln592*) | Deleterious | 1 | |
| NLRC5 | 11 | Base 23 del1 | c.2566del | Deleterious | 1 | |
| | | | (p.Val856Serfs*8) | | | |
| NLRC5 | 23 | Base 78 del1 | c.3584del | Deleterious | 1 | |
| | | | (p.Lys1195Argfs*38) | | | |
| NLRC5 | 36 | Base 23 G > T | c.4606G > T (p.Glu1536*) | Deleterious | 1 | |
| NLRC5 | 42 | Base 68 del1 | c.5149del | Deleterious | 1 | |
| | | | (p.His1717Ilefs*29) | | | |
| TAPBP | 4 | Base 92 del1 | c.561del | Deleterious | 2 | 20 |
| | | | (p.Thr188Profs*17), | | | |
| | | | c.300del | | | |
| | | | (p.Thr101Profs*17) | | | |
| B2M | 1 | Base 3 G > C | c.3G > C (p.Met1?) | Suspected | 1 | |
| | | | | Deleterious | | |
| B2M | 1 | Base 69 T > C | c.67+2T > C | Suspected | 1 | |
| | | | | Deleterious | | |
| B2M | 2 | Base 148 del12 | c.215_226del | Suspected | 1 | |
| | | | (p.Ser72_Ser75del) | Deleterious | | |
| B2M | 2 | Base 235 G > T | c.302G > T (p.Arg101Leu) | Suspected | 1 | |
| | | | | Deleterious | | |
| B2M | 2 | Base 250 del34 | c.317_346+4del | Suspected | 2 | |
| | | | | Deleterious | | |
| CIITA | 11 | Base 132 C > T | C.1138C > T (p.Arg380Trp), | Suspected | 1 | |
| | | | C.1141C > T (p.Arg381Trp) | Deleterious | | |
| CIITA | 11 | Base 322 C > G | C.1328C > G (p.Pro443Arg), | Suspected | 2 | |
| | | | C.1331C > G (p.Pro444Arg) | Deleterious | | |
| ERAP2 | 6 | Base 107 T > G | c.1232T > G (p.Leu411Arg) | Suspected | 10 | |
| | | | | Deleterious | | |
| ERAP2 | 14 | Base 82 C > T | C.2251C > T (p.Arg751Cys) | Suspected | 7 | |
| | | | | Deleterious | | |
| NLRC5 | 17 | Base 69 C > A | C.3098C > A (p.Ala1033Asp) | Suspected | 2 | |
| | | | | Deleterious | | |
| NLRC5 | 22 | Base 56 C > T | c.3478C > T (p.Pro1160Ser) | Suspected | 1 | |
| | | | | Deleterious | | |
| NLRC5 | 36 | Base 30 G > A | c.4613G > A (p.Gly1538Asp) | Suspected | 1 | |
| | | | | Deleterious | | |
| NLRC5 | 47 | Base −1 G > A | C.5490−1G > A | Suspected | 1 | |
| | | | | Deleterious | | |
| TAP1 | 7 | Base 170 C > T | c.1727C > T (p.Pro576Leu), | Suspected | 1 | |
| | | | c.944C > T (p.Pro315Leu) | Deleterious | | |
| TAP1 | 7 | Base 188 A > C | c.1745A > C (p.Gln582Pro), | Suspected | 1 | |
| | | | c.962A > C (p.Gln321Pro) | Deleterious | | |
| TAP1 | 10 | Base 40 G > A | c.2123G > A (p.Arg708Gln), | Suspected | 29 | |
| | | | c.1340G > A (p.Arg447Gln) | Deleterious | | |
| TAP2 | 3 | Base −1 G > A | c.609−1G > A | Suspected | 1 | |
| | | | | Deleterious | | |
| TAP2 | 4 | Base 199 G > A | c.938G > A (p.Arg313His) | Suspected | 8 | |
| | | | | Deleterious | | |
| TAP2 | 7 | Base 127 del1 | c.1399del | Suspected | 1 | |
| | | | (p.Val467Leufs*2) | Deleterious | | |
| TAPBP | 2 | Base 66 G > A | c.103G > A (p.Gly35Arg) | Suspected | 2 | |
| | | | | Deleterious | | |
| TAPBP | 2 | Base 124 C > G | C.161C > G (p.Pro54Arg) | Suspected | 1 | |
| | | | | Deleterious | | |
| TAPBP | 2 | Base 137 | c.174_175delinsTT | Suspected | 12 | |
| | | del2insTT | (p.Asp59delinsTyr) | Deleterious | | |
| TAPBP | 4 | Base 261 | c.730_731delinsAT | Suspected | 1 | 88 |
| | | del2insAT | (p.Asp244delinsIle), | Deleterious | | |
| | | | c.469_470delinsAT | | | |
| | | | (p.Asp157delinsIle) | | | |

| | |
|---|---|
| Total | 108 |

TABLE F

| | | Suspected |
|---|---|---|
| | Deleterious | Deleterious |
| Gene | Count | Count |
| B2M | 3 | 5 |
| CITTA | 1 | 2 |
| ERAP1 | 1 | |
| ERAP2 | 2 | 2 |
| NLRC5 | 8 | 4 |
| TAP1 | | 3 |
| TAP2 | | 3 |
| TAPBP | 1 | 4 |

Between 10% and 30% of tumors, depending on tissue and subtype, carried a mutation in at least one APM gene (Table G). Although overall mutation rates are similar, the number and nature of the mutated genes differed by tissue. Invasive ductal breast carcinoma (IDC) showed enrichment in mutations in the transporter associated with antigen processing (TAP1) gene, while colon adenocarcinomas presented more frequently with mutations in the MHC class I subunit beta2-microglobulin (B2M) and the MHC class I master transcriptional regulator NLRC5 (Table H).

TABLE G
Number/Frequency of Mutated Samples
by Tissue and Tissue Subtype

| | APM | | APM | APM non- |
|---|---|---|---|---|
| | deficient | | deficient | deficient |
| | (N) | Total | (%) | (%) |
| tissue | | | | |
| brain | 2 | 9 | 22.22 | 77.78 |
| breast | 26 | 155 | 16.77 | 83.23 |
| colorectal | 15 | 64 | 23.44 | 76.56 |
| lung | 39 | 226 | 17.26 | 82.74 |
| endometrial | 1 | 14 | 7.14 | 92.86 |
| ovary | 3 | 15 | 20.00 | 80.00 |
| | 86 | 483 | | |
| subtype | | | | |
| GBM | 2 | 9 | 22.22 | 77.78 |
| IDC | 24 | 144 | 16.67 | 83.33 |
| ILC | 2 | 11 | 18.18 | 81.82 |
| colonADC | 15 | 64 | 23.44 | 76.56 |
| ADSQ | 0 | 2 | 0.00 | 100.00 |
| CARC | 2 | 20 | 10.00 | 90.00 |
| LCC | 2 | 7 | 28.57 | 71.43 |
| LCNEC | 1 | 11 | 9.09 | 90.91 |
| lungADC | 15 | 73 | 20.55 | 79.45 |
| lungSCC | 17 | 79 | 21.52 | 78.48 |
| NSCLC | 0 | 26 | 0.00 | 100.00 |
| SCLC | 2 | 8 | 25.00 | 75.00 |
| EC | 1 | 14 | 7.14 | 92.86 |
| OC | 3 | 15 | 20.00 | 80.00 |
| Totals | 86 | 483 | | |

TABLE H
Mutations by Gene and Tissue Subtype

| tissue | subtype | B2M | CIITA | ERAP1 | ERAP2 | NLRC5 | TAP1 | TAP2 | TAPBP | total |
|---|---|---|---|---|---|---|---|---|---|---|
| breast | IDC | 0 | 0 | 0 | 6 | 1 | 15 | 4 | 5 | 31 |
| breast | ILC | 0 | 0 | 0 | 1 | 0 | 1 | 0 | 0 | 2 |
| lung | lungADC | 1 | 1 | 0 | 3 | 3 | 3 | 2 | 3 | 16 |
| lung | lungSCC | 2 | 1 | 0 | 1 | 2 | 4 | 1 | 7 | 18 |
| lung | CARC | 2 | 0 | 0 | 1 | 0 | 0 | 0 | 0 | 3 |
| lung | LCC | 0 | 0 | 0 | 1 | 0 | 1 | 1 | 0 | 3 |
| lung | LCNEC | 0 | 0 | 0 | 1 | 0 | 0 | 0 | 0 | 1 |
| lung | SCLC | 0 | 0 | 0 | 0 | 0 | 1 | 1 | 0 | 2 |
| lung | ADSQ. | 0 | 0 | 0 | 0 | 0 | 0 | 0 | 0 | 0 |
| lung | NSCLC | 0 | 0 | 0 | 0 | 0 | 0 | 0 | 0 | 0 |
| colorectal | colonADC | 6 | 1 | 1 | 4 | 7 | 3 | 1 | 3 | 26 |
| ovary | OC | 0 | 0 | 0 | 1 | 0 | 2 | 0 | 0 | 3 |
| endometrial | EC | 1 | 0 | 0 | 0 | 0 | 0 | 0 | 0 | 1 |
| brain | GBM | 0 | 1 | 0 | 0 | 0 | 1 | 0 | 0 | 2 |

| | | | | | | | | | |
|---|---|---|---|---|---|---|---|---|---|
| Totals | 12 | 4 | 1 | 19 | 13 | 31 | 10 | 18 | 108 |

A subset of CRC samples did have microsatellite stability status inferred by sequencing data on MRE11A and RAD50 loci. Frequency of APM mutations was analyzed by MSI status. Four out of five microsatellite unstable (MSI) colon tumors had an APM mutation, in contrast to 5 of 27 microsatellite stable (MSS) colon cancers (Table I).

TABLE I

| APM Genes mutated in CRC Sampled by MSI Status | | | | | | | | | | | | |
|---|---|---|---|---|---|---|---|---|---|---|---|---|
| | | | | | | | | | | | | # Samples |
| | | | | | | | | | | | # Muts | with |
| | | | | | | | | | | | per | mutation |
| Sample | | | | | | | | | MSI | total | mutated | per MSI |
| ID # | B2M | CIITA | ERAP1 | ERAP2 | NLRC5 | TAP1 | TAP2 | TAPBP | Status | mut | sample | status |
| 476815 | 0 | 0 | 1 | 0 | 3 | 0 | 0 | 0 | MSS | 4 | | |
| 477386 | 0 | 0 | 0 | 0 | 0 | 1 | 0 | 0 | MSS | 1 | 7 muts | 4 of 5 MSI |
| | | | | | | | | | | | in 4 | samples |
| | | | | | | | | | | | samples | have muts |
| 477387 | 0 | 0 | 0 | 0 | 0 | 0 | 0 | 1 | MSI | 1 | | 0.8 |
| 477388 | 0 | 0 | 0 | 0 | 0 | 0 | 0 | 1 | MSI | 1 | | |
| 477392 | 0 | 0 | 0 | 0 | 1 | 0 | 0 | 0 | MSS | 1 | | |
| 477393 | 0 | 1 | 0 | 0 | 2 | 0 | 0 | 1 | MSI | 4 | | |
| 477397 | 0 | 0 | 0 | 1 | 0 | 1 | 1 | 0 | MSS | 3 | 11 muts | 5 of 27 |
| | | | | | | | | | | | in 5 | MSS |
| | | | | | | | | | | | samples | samples |
| | | | | | | | | | | | | have muts |
| 477402 | 2 | 0 | 0 | 0 | 0 | 0 | 0 | 0 | MSS | 2 | | 0.19 |
| 477406 | 1 | 0 | 0 | 0 | 0 | 0 | 0 | 0 | MSI | 1 | | |
| 488295 | 1 | 0 | 0 | 0 | 0 | 0 | 0 | 0 | NA | 1 | | |
| 488656 | 2 | 0 | 0 | 0 | 0 | 0 | 0 | 0 | NA | 2 | 8 muts | 6 of 32 |
| | | | | | | | | | | | in 6 | with |
| | | | | | | | | | | | samples | unknown |
| | | | | | | | | | | | | MSI status |
| | | | | | | | | | | | | have |
| | | | | | | | | | | | | mutations |
| 488658 | 0 | 0 | 0 | 1 | 0 | 0 | 0 | 0 | NA | 1 | | 0.19 |
| 488659 | 0 | 0 | 0 | 1 | 0 | 0 | 0 | 0 | NA | 1 | | |
| 488661 | 0 | 0 | 0 | 1 | 1 | 0 | 0 | 0 | NA | 2 | | |
| 488666 | 0 | 0 | 0 | 0 | 0 | 1 | 0 | 0 | NA | 1 | | |
| CRC | MSI | MSS | NA | | | | | | | | | |
| N | 5 | 27 | 32 | 64 | | | | | | | | |
| APM | 4 | 5 | 6 | | | | | | | | | |
| deficient | | | | | | | | | | | | |
| APM | 1 | 22 | 26 | | | | | | | | | |
| normal | | | | | | | | | | | | |
| APM def | 80 | 19 | 19 | | | | | | | | | |
| (%) | | | | | | | | | | | | |

Without wishing to be bound by theory, MSI status has been described as a potential predictor of response to immune checkpoint inhibitors (ICI). The putative expression of many MSI-generated aberrant peptides may activate an immune response that can be unleashed by the application of ICI (Le et al., ONCOLOGIST (2016) 21:1200-1211). However, the same process may also increase the likelihood of mutations in APM genes, which in turn, as suggested by the present disclosure, may provide a selective advantage by immune escape for MSI clones with defective antigen presentation. Such clones may preexist as a minority in a heterogeneous tumor and lead to long-term resistance and recurrence after initially successful treatment with ICI. While colorectal carcinoma samples, especially microsatellite unstable CRC, show frequent mutations directly affecting the expression (NLRC5) or assembly (B2M) of MHC class I complexes, invasive breast cancer tumors are remarkable for mutations affecting peptide antigen processing. The most frequent variants in IDC were found in ERAP2 and the TAP complex (TAP1, TAP2, TAPBP). The TAP complex transports a wide range of proteasome generated peptides from the cytoplasma into the ER. ERAP2 is an endoplasmatic reticulum (ER) based aminopeptidase which processes medium-sized peptides (as transported by TAP) into the correct length for loading onto MHC class I heterodimers inside the ER. Modification of activity and/or specificity of TAP and/or ERAP2 may change the antigen peptide repertoire presented by MHC class I on the cell surface. For tumors with lower mutations rates (and fewer “aberrant” peptides), more subtle changes in peptide processing may suffice to avoid exposure of such neo-antigens at the cell surface.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference. The mere mentioning of the publications and patent applications does not necessarily constitute an admission that they are prior art to the instant application.

Although the foregoing disclosure has been described in some detail by way of illustration and example for purposes of clarity of understanding, it will be obvious that certain changes and modifications may be practiced within the scope of the appended claims.

1-21. (canceled)

22. A method comprising:
(1) assaying one or more human patient samples comprising genomic DNA isolated from a cancer cell to detect the sequence of at least a portion of each test gene in a panel of genes comprising the following antigen processing machinery genes: Beta-2-Microglobulin (B2M), Class II Major Histocompatibility Complex Transactivator (CIITA), Endoplasmic Reticulum Aminopeptidase 1 (ERAP1), Endoplasmic Reticulum Aminopeptidase 2 (ERAP2), NLR family CARD domain containing 5 (NLRC5), Transporter 1, ATP Binding Cassette Subfamily B Member (TAP1), Transporter 1, ATP Binding Cassette Subfamily B Member (TAP2), and TAP binding protein (TAPBP);
(2) determining whether any of the test genes harbors a mutation selected from the mutations listed in Table 4.

23. The method of claim 22, wherein the cancer is melanoma, renal cancer, lung cancer, bladder cancer, breast cancer, gastric cancer, prostate cancer, head and neck squamous cell carcinoma (HNSCC), or hematologic cancer.

24. The method of claim 22, wherein assaying in (1) comprises
(a) enriching genomic DNA of the one or more patient samples for target DNA fragments that collectively encompass all exons and 5 nucleotides upstream and downstream of each such exon of the tests genes and
(b) sequencing the target DNA fragments enriched in (1)(a).

25. The method of claim 22, wherein enriching in (1)(a) comprises either
(i)(A) hybridizing nucleic acid probes to target DNA fragments derived from the one or more patient samples, wherein the target DNA fragments collectively encompass all exons and 5 nucleotides upstream and downstream of each such exon of the tests genes, to separate the target DNA fragments from other DNA fragments derived from the one or more patient samples and
(i)(B) contacting the target DNA fragments with nucleic acid primers to amplify the target DNA fragments by polymerase chain reaction, or
(ii) contacting DNA derived from the one or more patient samples with nucleic acid primers that hybridize under stringent conditions with target DNA fragments collectively encompass all exons and 5 nucleotides upstream and downstream of each such exon of the tests genes to amplify the target DNA fragments by polymerase chain reaction.

26. The method of claim 22, further comprising determining microsatellite stability status of said cancer cell.

27. The method of claim 22, wherein the panel of genes further comprises at least one additional gene selected from: Major Histocompatibility Complex, Class I, A (HLA-A), Major Histocompatibility Complex, Class I, B (HLA-B), Major Histocompatibility Complex, Class I, C (HLA-C), Major Histocompatibility Complex, Class I, E (HLA-E), Major Histocompatibility Complex, Class I, G (HLA-G), or Protein Disulfide Isomerase Family A Member 3 (PDIA3).

28. The method of claim 22, wherein the sequences of the test genes are detected by Sanger sequencing, sequencing by synthesis, or single-molecule sequencing.

29. A system for detecting resistance to a treatment regimen comprising an immune checkpoint inhibitor, the system comprising:
(1) a sample analyzer for assaying one or more patient samples comprising or derived from a cancer cell to determine the sequence of at least a portion of each test gene in a panel of genes comprising at least 3 test genes selected from the genes listed in Table 1, wherein the sample analyzer contains the sample or DNA molecules extracted or derived from the sample;
(2) a first computer program for receiving test gene sequence data on the test genes;
(3) a second computer program for comparing the test gene sequence data to one or more reference gene sequences for each test gene to determine whether any of the test genes harbors a mutation; and
(4) a third computer program for determining
(a) that a patient in whose sample at least one test gene is determined by the second computer program in (3) to have a mutation has an increased likelihood of resistance to a treatment regimen comprising an immune checkpoint inhibitor or
(b) that a patient in whose sample no test gene is determined by the second computer program in (2) to have a mutation has a decreased likelihood of resistance to a treatment regimen comprising an immune checkpoint inhibitor.

30. A kit for detecting resistance to a treatment regimen comprising an immune checkpoint inhibitor, the kit comprising, in a compartmentalized container, a plurality of oligonucleotides hybridizing to a panel of genes comprising at least 3 test genes selected from the genes listed in Table 1, wherein (a) the panel consists of no more than 100 genes, and (b) the test genes represent at 5% of the panel of genes.

31. The kit of claim 30, wherein the oligonucleotides are either (a) nucleic acid probes for hybridization with amplified DNA corresponding to the test genes under stringent conditions or (b) primers suitable for PCR amplification of the test genes.

32. The kit of claim 30, further comprising instructions for detecting resistance to a treatment regimen comprising an immune checkpoint inhibitor based at least in part on the presence or absence of mutations in the test genes.

33. The kit of claim 30, further comprising one or more computer software programs for detecting resistance to a treatment regimen comprising an immune checkpoint inhibitor based at least in part on the presence or absence of mutations in the test genes.

34. The kit of claim 33, wherein the computer software program is capable of communicating or instructing a computer to record in a tangible medium whether (a) a patient in whose sample a mutation is detected in at least one test gene has an increased likelihood of resistance to a treatment regimen comprising an immune checkpoint inhibitor or (b) a patient in whose sample no mutation is detected in any test gene has a decreased likelihood of resistance to a treatment regimen comprising an immune checkpoint inhibitor.
